(19) United States
(12) Patent Application Publication
(10) Pub. No.: US 2007/0149567 A1
(43) Pub. Date: Jun. 28, 2007

(54) Quinoline compounds

(75) Inventors: Mary Didiuk, Madison, CT (US); Ryan Kelley, Groton, CT (US); David A. Perry, Mystic, CT (US); Roger Ruggeri, Waterford, CT (US)

Correspondence Address: PFIZER INC., PATENT DEPARTMENT, MS8260-1611, EASTERN POINT ROAD, GROTON, CT, 06340, US

(73) Assignee: Pfizer Inc, Groton, CT (US)

(21) Appl. No.: 10/576,853

(22) PCT Filed: Sep. 12, 2005
(86) PCT No.: PCT/IB05/02890

§ 371 Date: Apr. 20, 2006

Related U.S. Application Data
(60) Provisional application No. 60/612,863, filed on Sep. 23, 2004.

(51) Int. Cl. C07D 215/38; A61K 31/4706

(52) U.S. Cl. 514/313; 546/159

(57) ABSTRACT

Quinoline compounds, pharmaceutical compositions containing such compounds and the use of such compounds to elevate certain plasma lipid levels, including high density lipoprotein-cholesterol and to lower certain other plasma lipid levels, such as LDL-cholesterol and triglycerides and accordingly to treat diseases which are exacerbated by low levels of HDL cholesterol and/or high levels of LDLcholesterol and triglycerides, such as atherosclerosis and cardiovascular diseases in some mammals, including humans.

### BACKGROUND OF INVENTION

This invention relates to quinoline compounds, pharmaceutical compositions containing such inhibitors and the use of such inhibitors to elevate certain plasma lipid levels, including high density lipoprotein (HDL)-cholesterol and to lower certain other plasma lipid levels, such as low density lipoprotein (LDL)-cholesterol and triglycerides and accordingly to treat diseases which are affected by low levels of HbL cholesterol and/or high levels of LDL-cholesterol and triglycerides, such as atherosclerosis and cardiovascular diseases in certain mammals (i.e., those which have CETP in their plasma), including humans.

Atherosclerosis and its associated coronary artery disease (CAD) is the leading cause of mortality in the industrialized world. Despite attempts to modify secondary risk factors (smoking, obesity, lack of exercise) and treatment of dyslipidemia with dietary modification and drug therapy, coronary heart disease (CHD) remains the most common cause of death in the U.S., where cardiovascular disease accounts for 44% of all deaths, with 53% of these associated with atherosclerotic coronary heart disease.

Risk for development of this condition has been shown to be strongly correlated with certain plasma lipid levels. While elevated LDL-C may be the most recognized form of dyslipidemia, it is by no means the only significant lipid associated contributor to CHD. Low HDL-C is also a known risk factor for CHD (Gordon, D. J., et al.,: “High-density Lipoprotein Cholesterol and Cardiovascular Disease”, Circulation, (1989), 79: 8-15).

High LDL-cholesterol and triglyceride levels are positively correlated, while high levels of HDL-cholesterol are negatively correlated with the risk for developing cardiovascular diseases. Thus, dyslipidemia is not a unitary risk profile for CHD but may be comprised of one or more lipid aberrations.

Among the many factors controlling plasma levels of these disease dependent principles, cholesteryl ester transfer protein (CETP) activity affects all three. The role of this 70,000 dalton plasma glycoprotein found in a number of animal species, induding humans, is to transfer cholesteryl ester and triglyceride between lipoprotein particles, including high density lipoproteins (HDL), low density lipoproteins (LDL), very low density lipoproteins (VLDL), and chylomicrons. The net result of CETP activity is a lowering of HDL cholesterol and an increase in LDL cholesterol. This effect on lipoprotein profile is believed to be pro-atherogenic, especially in subjects whose lipid profile constitutes an increased risk for CHD.

No wholly satisfactory HDL-elevating therapies are on the market today. Niacin can significantly increase HDL, but has serious toleration issues which reduce compliance. Fibrates and the HMG CoA reductase inhibitors raise HDL-C, but in some patients, the result is an increase of modest porportions (˜10-12%). As a result, there is an unmet medical need for an approved therapeutic agent that elevates plasma HDL levels, thereby reversing or slowing the progression of atherosclerosis.

Thus, although there are a variety of anti-atherosclerosis therapies, there is a continuing need and a continuing search in this field of art for alternative therapies.

### SUMMARY OF THE INVENTION

This invention is directed to compounds selected from the group:

ˆ[AminoˆS.S-bis-trifluoromethyl-phenyO-methylJˆ-ethyl-e-trifluorornethyl-Sˆ-dihydroˆW-quinoline-1-carboxylic acid isopropyl ester;

(2R₁AR,4aS)-4-[Amino-(3,5-bis-trifluoromethyl-phenyl)-methyl]-2-ethyl-6-trifluoromethyl-3,4-dihydro-2H-quinoline-1-carboxylic acid isopropyl ester;

(2R,AR,4aR)-4-[Amino-(3,5-bis-trifluoromethyl-phenyl)-methyl]-2-ethyl-6-trifluoromethyl-3,4-dihydro-2W-quinoline-1-carboxylic acid isopropyl ester,

(2R,AS,4aS)-4-[Amino-(3,5-bis-trifluoromethyl-phenyl)-methyl]-2-ethyl-6-trifluoromethyl-3,4-dihydroˆH-quinoline-i-carboxylic acid isdpropyl ester;

(2R,AS,4aR)-4-[Amino-(3,5-bis-trifluoromethyl-phenyl)-methyl]-2-ethyl-6-trifluoromethyl-3,4-dihydro-2/-/-quinoline-1-carboxylic acid isopropyl ester;

4-[Amino-(3,5-bis(trifluoromethyl-phenyl)-methyl]-2-ethyl-6-trifluoromethyl-3,4-dihydro-2H-quinoline-1-carboxylic acid n-propyl ester,

(2R,AR,4aSH -[Amino-3,δ-bisOrifluoromethyl-phenyO-methyllˆ-ethyl-e-trifluoromethyl-Sˆ-dihydro-2W-quiπoline-1-carboxylic acid n-propyl ester;

(2R,4R,4aR)-4-[Amino-(3,5-bis(trifluoromethyl-phenyl)-methyl]-2-ethyl-6-trifluoromethyl-3,4-dihydroˆY-quinoline-i-carboxylic acid n-propyl ester;

(2R,AS,4aS)-4-[Amino-(3,5-bis(trifluoromethyl-phenyl)-methyl]-2-ethyl-6-trifluoromethyl-3,4-dihydroˆW-quinoline-i-carboxylic acid n-propyl ester;

(2R,AS,4aR)-4-[Amino-(3,5-bis(trifluoromethyl-phenyl)-methyl]-2-ethyl-6-trifluoromethyl-3,4-dihydroˆW-quinoline-i-carboxylicacid n-propyl ester;

4-[Amino-(3,5-bis(trifluoromethyl-phenyl)-methyl]-2-ethyl-6-trifluoromethyl-3,4-diriydro-2/-/-quinoline-1-carboxylic acid isobutyl ester;

(2R,4R,4aS)-4-[Amino-(3,5-bis(trifluoromethyl-phenyl)-methyl]-2-ethyl-6-trifluoromethyl-3,4-dihydroˆH-quinoline-i-carboxylic acid isobutyl ester,

(2R,4R,4aR)-4-[Amino-(3,5-bis(trifluoromethyl-phenyl)-methyl]-2-ethyl-6-trifluoromethyl-3,4-dihydroˆH-quinoline-i-carboxylic acid isobutyl ester;

(2R,AS,4aS)-4-[Amino-(3,5-bis(trifluoromethyl-phenyl)-methyl]-2-ethyl-6-trifluoromethyl-3,4-dihydroˆH-quinoline-i-carboxylic acid isobutyl ester,

(2R,AS,4aR)-4-tAmino-(3,5-bis(trifluoromethyl-phenyl)-methyl]-2-ethyl-6-trifluoromethyl-3,4-dihydroˆH-quinoline-i-carboxylic add isobutyl ester,

4-[Amino-(3,5-bis(trifluoromethyl-phenyl)-methyl]-2-cyclopropyl-6-trifluoromethyl-3,4-dihydro-2Ay-quinoline-1-carboxylic acid isopropyl ester;

(2S,4R,4aS)-4-[Amino-(3,5-bis(trifluoromethyl-phenyl)-methyl]-2-cyclopropyl-6-trifluoromethyl-3,4-dihydro-2H-quinoline-1-carboxylic acid isopropyl ester;

(2S,4R,4aR)-4-[Amino-(3,5-bis(trifluoromethyl-phenyl)-methyl]-2-cyclopropyl-6-trifluoromethyl-3,4-dihydro-2H-quinoline-1-carboxylic acid isopropyl ester,

(2S,4S,4aS)-4-[Amino-(3,5-bis(trifluoromethyl-phenyl)-methyl]-2-cyclopropyl-6-trifluoromethyl-3,4-dihydro-2H-quinoline-1-carboxylic acid isopropyl ester;

(2S,4S,4aR)-4-[Amino-(3,5-bis(trifluoromethyl-phenyl)-methyl]-2-cyclopropyl-6-trifluoromethyl-3,4-dihydro-2W-quinoIine-1-carboxylic acid isopropyl ester,

4-[Amino-(3,5-bis(trifluoromethyl-phenyl)-methyl]-2-methyl-6-trifluoromethyl-3,4-dihydro-2H-quinoline-1-carboxylic acid ethyl ester;

(2R,AR,4a S)-4-[A mino-(3,5-bisˆrifluoromethyl-phenyO-metriyll-Z-methyl-e-trifluoromethyl-Sˆ-dihydro-2AV-quinoline-1-carboxylic acid ethyl ester;

(2R,AR,4aR)-4-tAmino-(3,5-bis(trifluoromethyl-phenyl)-methyl]-2-methyl-6-trifluoromethyl-3,4-dihydro-2/-/-quinoline-1-carboxylic acid ethyl ester;

(2R,AS,4aS)-4-[Amino-(3,5-bis(trifluoromethyl-phenyl)-methyl]-2-methyl-trifluoromethyl-3,4-dihydro-2W-quinoline-1-carboxylic acid ethyl ester;

(2R,AS,4aR)-4-[Amino-(3,5-bis(trifluoromethyl-phenyl)-methyl]-2-methyl-6-trifluoromethyl-3,4-dihydro-2H-quinoline-1-carboxylic acid ethyl ester;

4-[Amino-(3,5-bis(trifluoromethyl-phenyl)-methyl]-2-methyl-6-trifluoromethyl-3,4-dihydro-2H-quinoline-1-carboxylic acid isopropyl ester;

(2R,AR,4aS)-4-[Amino-(3,5-bis(trifluoromethyl-phenyl)-methyl]-2-methyl-6-trifluoromethyl-3,4-dihydro-2/-/-quinoline-1-carboxylic acid isopropyl ester;

(2R,4R,4aR)-4-[Amino-(3,5-bis(trifluoromethyl-phenyl)-rnethyl]-2-methyl-6-trifluoromethyl-3,4-dihydro-2/-/-quinoline-1-carboxylic acid isopropyl ester;

(2R,AS,4aS)-4-[Amino-(3,5-bis(trifluoromethyl-phenyl)-methyl]-2-methyl-6-trifluoromethy-3,4-dihydro-2H-quinoline-1-carboxylic acid isopropyl ester;

(2R,AS,4aR)-4-[Amino-(3,5-bis(trifluoromethyl-phenyl)-methyl]-2-methyl-trifluoromethyl-3,4-dihydro-2/-/-quinoline-1-carboxylic acid isopropyl ester;

4-[Amino-(3,5-bis(trifluoromethyl-phenyl)-methyl]-2-methyl-6-trifluoromethyl-3,4-dihydro-2/-/-quinoline-1-carboxylic acid n-propyl ester;

(2R,AR,4aS)-4-[Amino-(3,5-bis(trifluoromethyl-phenyl)-methyl]-2-methyl-trifluoromethyl-3,4-dihydro-2H-quinoline-1-carboxylic acid n-propyl ester;

(2R₁AR,4aR)-4-[Amino-(3,5-bis(trifluoromethyl-phenyl)-methyl]-2-methyl-6-trifluoromethyl-3,4-dihydro-2H-quinoline-1-carboxylic acid n-propyl ester;

(2R,AS,4aS)-4-[Amino-(3,5-bis(trifluoromethyl-phenyl)-methyl]-2-methyl-6-trifluoromethyl-3,4-dihydro-2/-/-quinoline-1-carboxylic acid n-propyl ester;

(2R,AS,4aR)-4-[Amino-(3,5-bis(trifluoromethyl-phenyl)-methyl]-2-methyl-6-trifluoromethyl-3,4-dihydro-2/-/-quinoline-1-carboxylic acid n-propyl ester;

4-[Amino-(3,5-bis(trifluoromethyl-phenyl)-methyl]-2-methyl-6-trifluoromethyl-3,4-dihydro-2H-quinoline-1-carboxylic acid f-butyl ester;

(2R₁AR,4aS)-4-[Amino-(3,5-bis(trifluoromethyl-phenyl)-methyl]-2-methyl-6-trifluoromethyl-3,4-dihydro-2H-quinoline-1-carboxylic acid f-butyl ester;

(2R,AR,4aR)-4-[Amino-(3,5-bis(trifluoromethyl-phenyl)-methyl]-2-methyl-6-trifluoromethyl-3,4-dihydro-2H-quinoline-1-carboxylic acid f-butyl ester;

(2R,AS,4aS)-4-[Amino-(3,5-bis(trifluoromethyl-phenyl)-methyl]-2-methyl-6-trifluoromethyl-3,4-dihydro-2W-quinoline-1-carboxylic acid f-butyl ester;

(2R,AS,4aR)-4-[Amino-(3,5-bis(trifluoromethyl-phenyl)-methyl]-2-methyl-6-trifluoromethyl-3,4-dihydro-2H-quinoline-1-carboxylic acid f-butyl ester;

4-[Amino-(3,5bis(chloro-phenyl)-methyl]-2-ethyl-6-trifluoromethyl-3,4-dihydro-2H-quinoline-1-carboxylic acid ethyl ester;

(2R,4R,4aS)-4-[Amino-(3,5bis(chloro-phenyl)-methyl]-2-ethyl-6-trifluoromethyl-3,4-dihydro-2H-quinoline-1-carboxylic acid ethyl ester;

(2R,AR,4af?)-4-[Amino-(3,5bis(chloro-phenyl)-methyl]-2-ethyl-6-trifluoromethyl-3,4-dihydro-2H-quinoline-1-carboxylic acid ethyl ester;

(2R,4S,4aS)-4-[Amino-(3,5-bis(chloro-phenyl)-methyl]-2-ethyl-6-trifluoromethyl-3,4-dihydro-2H-quinoline-1-carboxylic acid ethyl ester;

(2R,4S,4aR)-4-[Amino-(3,5-bis(chloro-phenyl)-methyl]-2-ethyl-6-trifluoromethyl-3,4-dihydro-2H-quinoline-1-carboxylic acid ethyl ester;

4-[Amino-(3-chloro-5-trifluoromethyl-phenyl)-methyl]-2-ethyl-6-trifluoromethyl-3,4-dihydro-2H-quinoline-1-carboxylic acid ethyl ester;

(2R₁AR,4aS)-4-[Amino-(3-chloro-5-triuoromethyl-phenyl)-methyl]-2-ethyl-6-trifluoromethyl-3,4-dihydro-2H-quinoline-1-carboxylic acid ethyl ester;

(2R4R,4aR)-4-[Amino-(3-chloro-5-trifluoromethyl-phenyl)-methyl]-2-ethyl-6-trifluoromethyl-3,4-dihydro-2H-quinoline-1-carboxylic acid ethyl ester;

(2R,AS,4aS)-4-[Amino-(3-chloro-5-trifluoromethyl-phenyl)-methyl]-2-ethyl-6-trifluoromethyl-3,4-dihydro-2W-quinoline-1-carboxylic acid ethyl ester; and

(2RAS, 4aR)-4-[Amino-(3-chloro-5-trifluoromethyl-phenyl)-methyl]-2-ethyl-6-trifluoromethyl-3,4-dihydro-2H-quinoline-1-carboxylic acid ethyl ester;

or a prodrug thereof, or a pharmaceutically acceptable salt of said compound or of said prodrug.

In addition, the present invention provides methods for treating atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal by administering to a mammal in need of such treatment an atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction treating amount of a compound of the present invention, or a pharmaceutically acceptable form of said compound.

In addition, the present invention provides pharmaceutical compositions which comprise a therapeutically effective amount of a compound of the present invention, or a pharmaceutically acceptable form of said compound and a pharmaceutically acceptable vehicle, diluent or carrier.

In addition, the present invention provides pharmaceutical compositions for the treatment of atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal which comprise a therapeutically effective amount of a compound of the present invention, or a pharmaceutically acceptable form of said compound and a pharmaceutically acceptable vehicle, diluent or carrier.

Moreover, the present invention provides pharmaceutical combination compositions comprising: a therapeutically effective amount of a composition comprising a first compound, said first compound being a compound of the present invention, or a pharmaceutically acceptable form of said compound;

a second compound, said second compound being an HMG CoA reductase inhibitor, an MTP/Apo B secretion inhibitor, a PPAR modulator, a bile acid reuptake inhibitor, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant (preferably an HMG-CoA reductase inhibitor, a PPAR modulator, lovastatin, simvastatin, pravastatin, fluvastatin, atorvastatin, rivastatin, rosuvastatin or pravastatin); and

a pharmaceutical vehicle, diluent or carrier. This composition may be used to treat the aforementioned diseases, including atherosclerosis.

Also, the present invention provides a kit for achieving a therapeutic effect in a mammal comprising packaged in association a first therapeutic agent comprising a therapeutically effective amount of a compound of claim 1, 8, 12, or 13, a prodrug thereof, ora pharmaceutically acceptable salt of said compound or of said prodrug and a pharmaceutically acceptable carrier, a second therapeutic agent comprising a therapeutically effective amount d an HMG CoA reductase inhibitor, a PPAR modulator, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant and a pharmaceutically acceptable carrier and directions for administration of said first and second agents to achieve the therapeutic effect.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of exemplary embodiments of the invention and the examples included therein.

Before the present compounds, compositions and methods are disclosed and described, it is to be understood that this invention is not limited to specific synthetic methods of making that may of course vary. It is also to be understood that the terminology used herein is for the purpose of describing particular embodiments only and is not intended to be limiting.

The present invention also relates to the pharmaceutically acceptable acid addition salts of compounds of the present invention. The acids which are used to prepare the pharmaceutically acceptable acid addition salts of the aforementioned base compounds of this invention are those which form non-toxic acid addition salts, (Le₁, salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The invention also relates to base addition salts of the compounds of the present invention. The chemical bases that may be used as reagents to prepare pharmaceutically acceptable base salts of those compounds of the present invention that are acidic in nature are those that form non-toxic base salts with such compounds. Such non-toxic base salts include, but are not limited to those derived from such pharmacologically acceptable cations such as alkali metal cations (ag., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

The chemist of ordinary skill will recognize that certain compounds of this invention will contain one or more atoms which may be in a particular stereochemical or geometric configuration, giving rise to stereoisomers and configurational isomers. All such isomers and mixtures thereof are included in this invention. Hydrates and solvates of the compounds of this invention are also included.

Where the compounds of the present invention possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form.

The compounds of this invention may contain olefin-like double bonds. When such bonds are present, the compounds of the invention exist as cis and trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

Alpha and Beta refer to the orientation of a substituent with reference to the plane of the ring. Beta is above the plane of the ring and Alpha is below the plane of the ring.

This invention also includes isotopically-labeled compounds, which are identical to those described by formulas I and II, except for the fact that one or more atoms are replaced by one or more atoms having specific atomic mass or mass numbers. Examples of isotopes that can be incorporated into compounds of the invention include isotopes of hydrogen, carbon, nitrogen, oxygen, sulfur, fluorine, and chlorine such as ²H, ³H, ¹³C, ¹⁴C, ¹⁵N, ¹⁸O, ¹⁷O, ¹⁸F, and ³⁶Cl respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (Ae., ²H), can afford certain therapeutic advantages resulting from greater metabolic stability, for example increased in vivo half-life or reduced dosage requirements and, hence, may be preferred in some circumstances, Isotopically labeled compounds of this invention and prodrugs thereof can generally be prepared by carrying out the procedures disclosed in the schemes and/or in the Examples below, by substituting a readily available isotopically labeled reagent for a non-isotopically labeled reagent.

In this specification and in the claims that follow, reference will be made to a number of terms that shall be defined to have the following meanings:

As used herein, the term mammals is meant to refer to all mammals which contain CETP in their plasma, for example, rabbits and primates such as monkeys and humans, including males and females. Certain other mammals e.g., dogs, cats, cattle, goats, sheep and horses do not contain CETP in their plasma and so are not included herein.

The term “treating”, “treat” or “treatment” as used herein includes preventative (e.g., prophylactic) and palliative treatment.

By “pharmaceutically acceptable” is meant the carrier, diluent, excipients, and/or salt must be compatible with the other ingredients of the formulation, and not deleterious to the recipient thereof.

“Compounds” when used herein includes any pharmaceutically acceptable derivative or variation, including conformational isomers (ag., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. By “tautomers” is meant chemical compounds that may exist in two or more forms of different structure (isomers) in equilibrium, the forms differing, usually, in the position of a hydrogen atom. Various types of tautomerism can occur, including keto-enol, ring-chain and ring-ring tautomerism. The expression “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the compounds of the present invention include but are not limited to those having a carboxyl moiety wherein the free hydrogen is replaced by (CrC₄)alkyl, (C₂-C₇)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(CrC₂)alkylamino(C₂-C₃)alkyl (such as β-dimethylaminoethyl), carbamoyl-(CrC₂)alkyl, N,N-di(CrC₂)alkylcarbamoyl-(CrC₂)alkyl and piperidino-, pyrroiidino- or morpholino(C₂-C₃)alkyl.

The following paragraphs describe exemplary ring(s) for the generic ring descriptions contained herein.

By “halo” or “halogen” is meant chloro, bromo, iodo, or fluoro.

By “alkyl” is meant straight chain saturated hydrocarbon or branched chain saturated hydrocarbon. Exemplary of such alkyl groups (assuming the designated length encompasses the particular example) are methyl, ethyl, propyl, isopropyl, butyl, sec-butyl, tertiary butyl, isobutyl, pentyl, isopentyl, neopentyl, tertiary pentyl, 1-methylbutyl, 2-methylbutyl, 3-methylbutyl, hexyl, isohexyl, heptyl and octyl.

“Alkenyl” referred to herein may be linear or branched, and they may also be cyclic (e.g. cyclobutenyl, cyclopentenyl, cyclohexenyl) or bicyclic or contain cyclic groups. They contain 1-3 carbon-carbon double bonds, which can be cis or trans.

By “alkoxy” is meant straight chain saturated alkyl or branched chain saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy .

As used herein, the expressions “reaction-inert solvent” and “inert solvent” refer to a solvent or a mixture thereof which does not interact with starting materials, reagents, intermediates or products in a manner which adversely affects the yield of the desired product.

In one embodiment the present invention, the compound is selected from the group consisting of:

4-[Amino-(3,5-bis-trifluoromethyl-phenyl)-methyl]-2-ethyl-6-trifluoromethyl-3,4-dihydro-2H-quinoline-1-carboxylic acid isopropyl ester;

4-tAmino-(3,5-bis(trifluoromethyl-phenyl)-methyl]-2-ethyl-6-trifluoromethyl-3,4-dihydro-2H-quinoline-1-carboxylic acid n-propyl ester;

4-[Amino-(3,5-bis(trifluoromethyl-phenyl)-methyl]-2-ethyl-6-trifluoromethyl-3,4-dihydro-2H-quinoline-1-carboxylic add isobutyl ester;

4-[Amino-(3,5-bis(trifluoromethyl-phenyl)-methyl]-2-cyclopropyl-6-trifluoromethyl-3,4-dihydro-2H-quinoline-1-carboxylic add isopropyl ester;

4-[Amino-(3,5-bis(trifluoromethyl-phenyl)-methyl]-2-methyl-6-trifluoromethyl-3,4-dihydro-2Ay-quinoline-1-carboxylic acid ethyl ester;

4-[Amino-(3,5-bis(trifluoromethyl-phenyl)-methyl]-2-methyl-6-trifluoromethyl-3,4-dihydro-2W-quinoline-1-carboxylic acid isopropyl ester;

4-[Amino-(3,5-bis(trifluoromethyl-phenyl)-methyl]-2-methyl-6-trifluoromethyl-3,4-dihydro-2W-quinoline-1-carboxylic acid n-propyl ester;

4-[Amino-(3,5-bis(trifluoromethyl-phenyl)-methyl]-2-methyl-6-trifluoromethyl-3,4-dihydro-2H-quinoline-1-carboxylic acid f-butyl ester;

4-[Amino-(3,5-bis(chloro-phenyl)-methyl]-2-ethyl-6-trifluoromethyl-3,4-dihydro-2H-quinoline-1-carboxylic add ethyl ester; and

4-[Amino-(3-chloro-5-trifluoromethyl-phenyl)-methyl]-2-ethyl-6-trifluoromethyl-3,4-dihydro-2H-quinoline-1-carboxylic acid ethyl ester.

In another embodiment the present invention, the compound is selected from the group consisting of:

(2R,AR,4aS)-4-[Amino-(3,5-bis-trifluoromethyl-phenyl)-methyl]-2-ethyl-6-trifluoromethyl-3,4-dihydro-2/-/-quinoline-1-carboxylic acid isopropyl ester;

(2R,AR,4aS)-4-[Amino-(3,5-bis(trifluoromethyl-phenyl)-methyl]-2-ethyl-6-trifluoromethyl-3,4-dihydro-2H-quinoline-1-carboxylic acid n-propyl ester;

(2R,AR,4aS)-4-[Amino-(3,5-bis(trifluoromethyl-phenyl)-methyl]-2-ethyl-6-trifluoromethyl-3,4-dihydro-2H-quinoline-i-carboxylic acid isobutyl ester;

(2S,4R,4aS)-4-[Amino-(3,5-bis(trifluoromethyl-phenyl)-methyl]-2-cyclopropyl-6-trifluoromethyl-3,4-dihydro-2H-quinoline-1-carboxylic acid isopropyl ester;

(2R,AR,4aS)-4-[Amino-(3,5-bis(trifluoromethyl-phenyl)-methyl]-2-methyl-6-trifluoromethyl-3,4-dihydro-2H-quinoline-1-carboxylic acid ethyl ester;

(2R,AR,4aS)-4-[Amino-(3,5-bis(trifluoromethyl-phenyl)-methyl]-2-methyl-6-trifluoromethyl-3,4-dihydro-2/-/-quinoline-1-carboxylic acid isopropyl ester;

(2R₁4R,4aS)-4-[Amino-(3,5-bis(trifluoromethyl-phenyl)-methyl]-2-methyl-6-trifluoromethyl-3,4-dihydro-2H-quinoline-1-carboxylic acid n-propyl ester;

(2R,AR,4aS)-4-[Amino-(3,5-bis(trifluoromethyl-phenyl)-methyl]-2-methyl-6-trifluoromethyl-3,4-dihydro-2H-quinoline-1-carboxylic acid £-butyl ester;

(2R,AR,4aS)-4-[Amino-(3 5-bis(chloro-phenyl)-methyl]-2-ethy!-6-trifluoromethyl-3,4-dihydro-2H-quinoline-1-carboxylic acid ethyl ester; and

(2R,AR,4aS)-4-[Amino-(3-chloro-5-trifluoromethyl-phenyl)-methyl]-2-ethyl-6-trifluoromethyl-3,4-dihydro-2/-/-quinoline-1-carboxylic acid ethyl ester.

In one embodiment of the method of the present invention, atherosclerosis is treated.

In another embodiment of the method of the present invention, peripheral vascular disease is treated.

In another embodiment of the method of the present invention, dyslipidemia is treated.

In another embodiment of the method of the present invention, hyperbetalipoproteinemia is treated.

In another embodiment of the method of the present invention, hypoalphalipoproteinemia is treated.

In another embodiment of the method of the present invention, familial-hypercholesterolemia is treated.

In another embodiment of the method of the present invention, coronary artery disease is treated.

In another embodiment of the method of the present invention, myocardial infarction is treated.

In one embodiment of the combination or kit of the present invention, the second compound is an HMG-CoA reductase inhibitor or a PPAR modulator.

In another embodiment of the combination or kit of the present invention, the second compound is lovastatin, simvastatin, pravastatin, fluvastatin, atorvastatin, rivastatin, rosuvastatin or pravastatin.

In another embodiment of the combination or kit of the present invention, the combination further comprising a cholesterol absorption inhibitor, wherein the cholesterol absorption inhibitor may be ezetimibe.

In general, the compounds of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section. Analogous processes are disclosed in the following U.S. patents, which are hereby incorporated by reference herein in their entirety: U.S. Pat. No. 6,140,342; U.S. Pat. No. 6,362,198; U.S. Pat. No. 6,147,090; U.S. Pat. No. 6, 395,751; U.S. Pat. No. 6,147,089; U.S. Pat. No. 6,310,075; U.S. Pat. No. 6,197,786; U.S. Pat. No. 6,140,343; U.S. Pat. No. 6,489,478; and International Publication No. WO 00/17164.

The Reaction Schemes herein described are intended to provide a general description of the methodology employed in the preparation of many of the Examples given. However, it will be evident from the detailed descriptions given in the Experimental section that the modes of preparation employed extend further than the general procedures described herein. In particular, it is noted that the compounds prepared according to these Schemes may be modified further to provide new Examples within the scope of this invention. For example, an ester functionality may be reacted further using procedures well known to those skilled in the art to give another ester, an amide, a carbinol or a ketone.

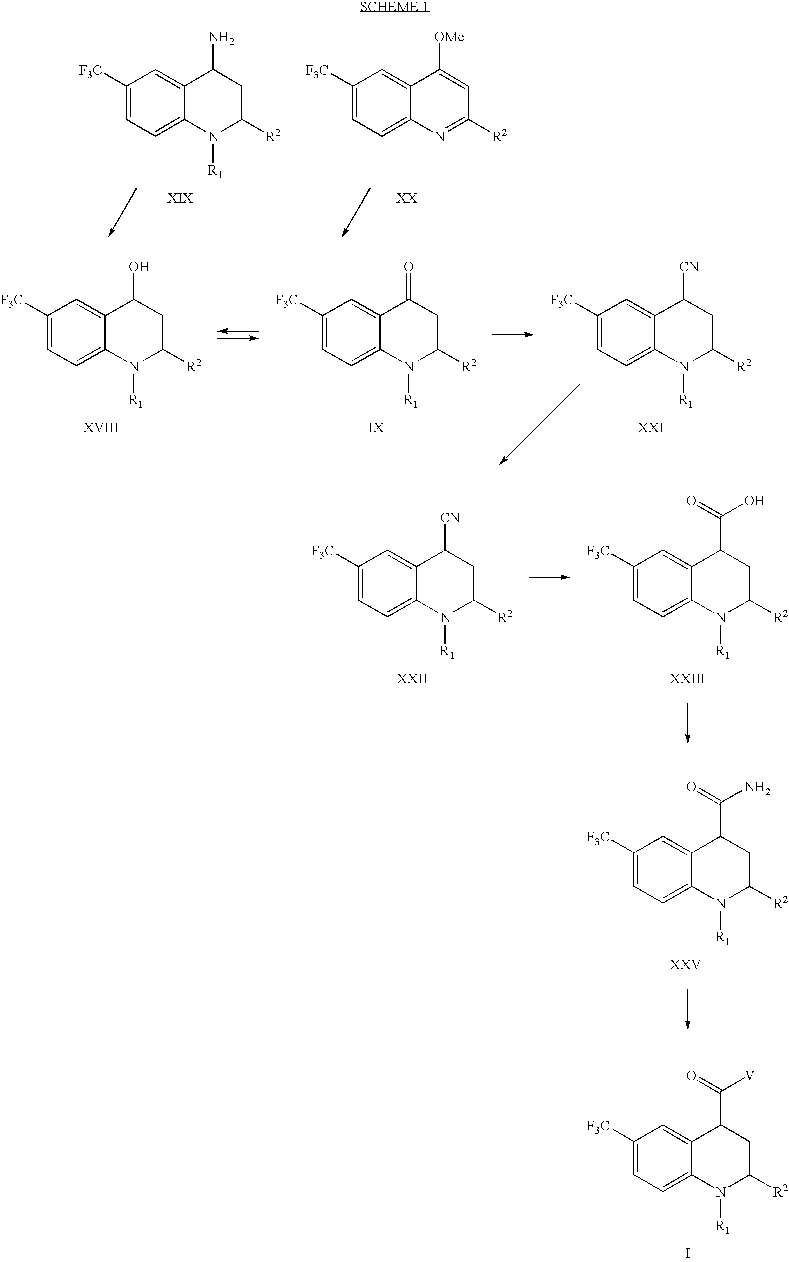

### Scheme 1

According to reaction Scheme 1, the desired compounds wherein R¹ is —COO(CᵣC₆)alkyl, R² is (Ci-Ce)alkyl or (C₃-C₆)cyclyoalkyl, Q is a leaving group such as chlorine, bromine, methanesulfonyloxy or p-toluenesulfonyloxy, and V is phenyl mono- or di-substituted with CF₃ or halo may be prepared from the corresponding Formula XXV by reduction with a hydride source, preferably sodium borohydride in an alcohol solvent such as methanol at a temperature of between 0 DC and 80 DC preferably 25 DC for 1 to 20 hours (preferably 1 hour). The mixture of diastereoisomeric alcohols produced may be separated chromatographically or carried forward as a mixture. The alcohol is converted to the mesylate by reaction with methanesulfonyl chloride at ambient temperature in methylene chloride in the presence of a suitable base such as triethylamine and the mesylate is then reacted with sodium azide in a reaction inert solvent such as dimethylformamide at a temperature of between 0 DC and 100 DC preferably 25-70 DC. The azide is converted to the primary amine by hydrogenation for example transfer hydrogenation with ammonium formate in the presence of palladium on carbon to give, after chromatographic separation of isomers if necesary, the desired Formula I compounds.

The desired Formula XXV compounds may be prepared from the corresponding Formula XXIII compounds by treating the acid in a reaction inert solvent (preferably dichloromethane) with N₁O-dimethylhydroxylamine in the presence of 1-hydroxybenzotriazole hydrate (HOBT) and 1-(3-dimethylaminopropyl)-3-ethylcarbodiimide hydrochloride (EDCl) at a temperature between ODC to 100 DC (preferably ambient temperature) for 1 to 24 hours (preferably 12 hours) to form the ‘Weinreb’ amide. This is reacted with an appropriate VMet compound where Met is a metal, preferably magnesium or lithium in a reaction inert solvent such as tetrahydrofuran or diethyl ether at a temperature of between −78 DC and 25 DC, preferably 0 DC to produce the desired Formula XXV compounds.

The desired Formula XXIII compounds of Scheme 1 may be prepared from the corresponding Formula XXII compounds by dissolving in concentrated sulfuric acid containing five equivalents of water at a temperature from 0° C. to 100° C. (preferably room temperature) for 1 to 20 hours. The resulting amide is then dissolved in a polar solvent (preferably methylene chloride) and treated with trimethyloxonium tetrafluoroborate at a temperature from 0° C. to 100° C. (preferably room temperature) for 1-20 hours (preferably 12 hours). The resulting imino ester is then treated with an aqueous base, preferably, lithium, sodium, or potassium hydroxide, in a polar solvent, preferably dioxane, at a temperature between 0° C. and 100° C. (preferably room temperature) for between 1 to 20 hours to provide the Formula XXIII Compounds.

The desired Formula XXII compounds of Scheme 1 may be prepared from the corresponding Formula XXI compounds by treatment with a reducing agent such as sodium borohydride or sodium cyanoborohydride in a reaction inert solvent such as methanol or ethanol, preferably ethanol, at a temperature of about 0° C. to about 100° C. (preferably reflux temperature) for 0.1 to 5 hours (preferably 0.75 hour) to provide the desired Formula XXII compounds.

Alternatively, the desired Formula XXII compounds may be prepared from the corresponding Formula XVII compounds, wherein Q is a leaving group as described above, by treatment with a cyanide salt such as lithium, sodium, potassium or a tetraalkylammonium cyanide in a reaction inert solvent such as dimethylformamide at a temperature between 0° C. to 100° C. for 1 to 12 hours, to provide the Formula XXII compounds.

The desired Formula XVII compounds of Scheme 1 wherein may be prepared as a mixture of diastereoisomers from the corresponding Formula XVIII compounds by reaction with the appropriate reagent such as methanesulfonyl chloride or toluenesulfonyl chloride in the presence of a suitable base such as diisopropylethylamine or triethylamine in a reaction inert solvent such as N,N-dimethylformamide, dimethylsulfoxide, chloroform, methylene chloride or toluene at a temperature between 0° C. to 60° C., typically ambient. Other suitable reagents for formation of the Formula XVII compounds include phosphorus (III) chloride, phosphorus (III) bromide and thionyl chloride optionally in a reaction inert solvent such as chloroform, methylene chloride, pyridine or toluene at a temperature between 0° C. to 60° C., typically ambient.

The desired Formula XVIII compounds of Scheme 1 may be prepared as a mixture of diastereoisomers from the corresponding Formula IX compounds by reduction of the carbonyl group using methods and reagents well known to those skilled in the arts, such as can be found in L.A. Paquette (Ed), Encyclopedia of Reagents for Organic Synthesis, John Wiley and Sons, Chichester, England, 1995, for example using sodium borohydride in an alcohol solvent such as methanol of ethanol at a temperature between 0° C. to 60° C., typically ambient or using potassium tri-sec-butylborohydride (K-Selectride®) in a reaction inert solvent such as tetrahydrofuran or diethyl ether at a temperature between −78° C. to 25° C., typically 0° C.

In an alternative procedure, the desired Formula XVIII compounds may be obtained by treatment of the corresponding Formula XIX compounds with sodium nitrite in the presence of an acid, preferably acetic add, followed by hydrolysis with a suitable base such as lithium, sodium, or potassium hydroxide, preferably sodium hydroxide in a suitable hydroxylic solvent such as ethanol to give the desired Formula XVIII compounds. Methods for the preparation of Formula XIX compounds are described in U.S. Pat. No. 6,197,786 and International Application WO 0140190.

The desired Formula IX compounds of Scheme 1 wherein R¹ is an alkoxycarbonyl group may be prepared from the corresponding 4-methoxyquinoline compounds of Formula XX by treatment with an organomagnesium derivative of the R² group together with an acylating agent such as ethyl chloroformate at a temperature between −100° C. to 70° C., typically −78° C. in a reaction inert solvent such as tetrahydrofuran followed by warming to a temperature between 0° C. and about 70° C. (preferably ambient) for between 0.1 and 24 hr, preferably lhr, followed by hydrolysis in aqueous acid, preferably 1N hydrochloric acid to give the desired Formula IX compounds, as described in U.S. Pat. No. 6,197,786.

In an alternative procedure, the desired Formula IX compounds may be obtained by oxidation of the corresponding Formula XVIII compounds using a variety of methods and reagents well known to those skilled in the arts, such as can be found in LA Paquette (Ed), Encyclopedia of Reagents for Organic Synthesis, John Wiley and Sons, Chichester, England, 1995, for example pyridinium chlorochromate, aqueous sodium hypochlorite in the presence of a catalytic amount of 2,2,6,6-tetramethyl-1-piperidinyloxy (TEMPO) free radical and catalytic potassium bromide in a suitable reaction inert solvent such as methylene chloride, or alternatively with acetic anhydride and dimethylsulfoxide.

The desired Formula XXI compounds of Scheme 1 may be prepared from the corresponding Formula IX compounds by treatment with trimethylsilylcyanide in an inert solvent such as an aromatic hydrocarbon (e.g.,benzene, toluene, xylene) in the presence of a catalytic amount of Lewis add, preferably zinc iodide, at a temperature of about 25° C. to about 140° C., preferably about 80° C. to about 100° C., for 1-12 hours, preferably 5 hours. The resulting solution is concentrated to dryness and added directly without further purification to a polar solvent (e.g., methanol, ethanol). A solution of acid (preferably hydrochloric) in a polar aprotic solvent (preferably dioxane) is added to the solution and the mixture is stirred at a temperature from 0° C. to about 100° C., preferably room temperature, for 1 to 24 hours, preferably 12 hours, to yield the Formula XXI compounds.

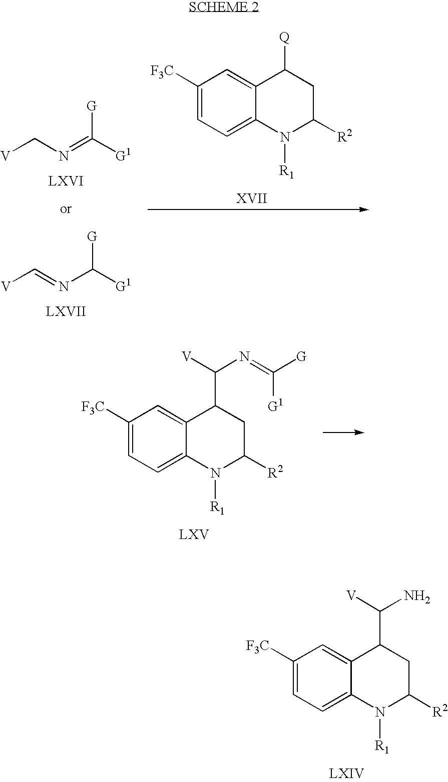

### Scheme 2

According to reaction Scheme 2, the desired compounds of Formula LXIV wherein R¹ is —COO(C₁-C₆)alkyl, R² is (C₁-C₆)alkyl or (C₃-C₆)cyclyoalkyl, and V is phenyl mono- or di-substituted with CF₃ or halo may be prepared as a mixture of diastereoisomers from the corresponding Formula LXV compounds by hydrolysis with an add such as hydrochloric acid or methanesulfonic add in the presence of water in a reaction inert solvent such as tetrahydrofuran, dioxane, isopropanol or diisopropyl ether at a temperature between 0° C. to 120° C., typically at reflux. The desired Formula LXIV compounds may be isolated as the salt by crystallization or converted to the free base by treatment with a base such as aqueous sodium hydroxide.

Alternatively, the imine LXV may be treated with a reagent such as hydroxylamine or hydrazine in the presence of water in a reaction inert solvent such as tetrahydrofuran, dioxane, isopropanol or diisopropyl ether at a temperature between 0° C. to 120° C., typically at reflux. This method typically produces a mixture of amine diastereomers LXIV that may be separated by silica gel chromatography.

The desired imine Formula LXV compounds wherein G is V or an aryl group such as phenyl and G¹ is either an aryl group such as phenyl or hydrogen in the case where G is V may be prepared as a mixture of diastereoisomers from the corresponding Formula XVII compounds where Q is a leaving group such as chlorine, bromine, methanesulfonyloxy or p-toluenesulfonyloxy, preferably chlorine or bromine by reaction with an imine of Formula LXVI or LXVII in the presence of a suitable base such as sodium hydride, sodium hexamethyldisilazide or potassium hexamethyldisilazide in a reaction inert solvent or mixture of solvents such as tetrahydrofuran, N,N-dimethylformamide or N-methylpyrrolidone at a temperature between −78° C. to 60° C., typically ambient.

The desired Formula LXVI compounds wherein V, G and G¹ are as described above may be prepared from the corresponding benzylamine VCH₂NH₂ by treatment with an aldehyde VCHO (when G is V and G¹ is H) or the ketone GC(O)G¹ or the imine GC(═NH)G¹ using methods and reagents well known to those skilled in the arts, such as can be found in L. A. Paquette (Ed), Encyclopedia of Reagents for Organic Synthesis, John Wiley and Sons, Chichester, England, 1995, for example using benzophenone imine in a reaction inert solvent such as diisopropyl ether or toluene at a temperature between 20° C. to 120° C., typically at reflux.

The desired Formula LXVII compounds wherein V, G and G¹ are as described above may be prepared from the corresponding aldehyde VCHO by treatment with an amine VCH₂NH₂ (when G is V and G¹ is H) or GCH(NH₂)G¹ under conditions where the water produced by the reaction can be continuously removed either by azeotropic distillation, for example in a Dean-Stark apparatus or by use of a drying agent such as anhydrous magnesium sulfate, in a reaction inert solvent such as toluene or diisopropyl ether at a temperature between 20° C. to 120° C., typically at reflux. Examples of alternative conditions are well known to those skilled in the arts and can be found in L. A. Paquette (Ed), Encyclopedia of Reagents for Organic Synthesis, John Wiley and Sons, Chichester, England, 1995.

As an initial note, in the preparation of compounds, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis. John Wiley & Sons, New York, 1991.

For example, in the reaction schemes, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the compound.

Prodrugs of the compounds of the present invention may be prepared according to methods known to those skilled in the art. Exemplary processes are described below.

Prodrugs of this invention where a carboxyl group in a carboxylic acid of the compounds is replaced by an ester may be prepared by combining the carboxylic acid with the appropriate alkyl halide in the presence of a base such as potassium carbonate in an inert solvent such as dimethylformamide at a temperature of about 0 to 100° C. for about 1 to about 24 hours. Alternatively the acid is combined with an appropriate alcohol as solvent in the presence of a catalytic amount of acid such as concentrated sulfuric acid at a temperature of about 20 to 100° C., preferably at a reflux, for about 1 hour to about 24 hours. Another method is the reaction of the acid with a stoichiometric amount of the alcohol in the presence of a catalytic amount of acid in an inert solvent such as toluene or tetrahydrofuran, with concomitant removal of the water being produced by physical (e.g., Dean-Stark trap) or chemical (e.g., molecular sieves) means.

Prodrugs of this invention where an alcohol function has been derivatized as an ether may be prepared by combining the alcohol with the appropriate alkyl bromide or iodide in the presence of a base such as potassium carbonate in an inert solvent such as dimethylformamide at a temperature of about 0 to 100° C. for about 1 to about 24 hours. Alkanoylaminomethyl ethers may be obtained by reaction of the alcohol with a bis-(alkanoylamino)methane in the presence of a catalytic amount of add in an inert solvent such as tetrahydrofuran, according to a method described in U.S. Pat. No. 4,997,984. Alternatively, these compounds may be prepared by the methods described by Hoffman et al. in J. Org. Chem. 1994, 59, 3530.

Glycosides are prepared by reaction of the alcohol and a carbohydrate in an inert solvent such as toluene in the presence of acid. Typically the water formed in the reaction is removed as it is being formed as described above. An alternate procedure is the reaction of the alcohol with a suitably protected glycosyl halide in the presence of base followed by deprotection.

N-(i-hydroxyalkyl) amides, N-1-hydroxy-1-(alkoxycarbonyl)methyl) amides may be prepared by the reaction of the parent amide with the appropriate aldehyde under neutral or basic conditions (e.g., sodium ethoxide in ethanol) at temperatures between 25 and 70° C. N-alkoxymethyl or N-1-(alkoxy)alkyl derivatives can be obtained by reaction of the N-unsubstituted compound with the necessary alkyl halide in the presence of a base in an inert solvent.

The compounds of this invention may also be used in conjunction with other pharmaceutical agents (e.g., LDL-cholesterol lowering agents, triglyceride lowering agents) for the treatment of the disease/conditions described herein. For example, they may be used in combination with a HMG-CoA reductase inhibitor, a cholesterol synthesis inhibitor, a cholesterol absorption inhibitor, another CETP inhibitor, a MTP/Apo B secretion inhibitor, a PPAR modulator and other cholesterol lowering agents such as a fibrate, niacin, an ion-exchange resin, an antioxidant, an ACAT inhibitor, and a bile acid sequestrant. Other pharmaceutical agents would also include the following: a bile acid reuptake inhibitor, an ileal bile acid transporter inhibitor, an ACC inhibitor, an antihypertensive (such as NORVASC®), a selective estrogen receptor modulator, a selective androgen receptor modulator, an antibiotic, an antidiabetic (such as metformin, a PPARy activator, a sulfonylurea, insulin, an aldose reductase inhibitor (ARI) and a sorbitol dehydrogenase inhibitor (SDI)), and aspirin (acetylsalicylic acid or a nitric oxide releasing asprin). A slow-release form of niacin is available and is known as Niaspan. Niacin may also be combined with other therapeutic agents such as statins, i.e. lovastatin, which is an HMG-CoA reductase inhibitor and described further below. This combination therapy is known as ADVICOR® (Kos Pharmaceuticals Inc.) In combination therapy treatment, both the compounds of this invention and the other drug therapies are administered to mammals (e.g., humans, male orfemale) by conventional methods.

Any HMG-CoA reductase inhibitor may be used in the combination aspect of this invention. The term HMG-CoA reductase inhibitor refers to compounds which inhibit the bioconversion of hydroxymethylglutaryl-coenzyme A to mevalonic acid catalyzed by the enzyme HMG-CoA reductase. Such inhibition is readily determined by those skilled in the art according to standard assays (e.g., Meth. Enzymol. 1981; 71:455-509 and references cited therein). A variety of these compounds are described and referenced below however other HMG-CoA reductase inhibitors will be known to those skilled in the art. U.S. Pat. No. 4,231,938 (the disclosure of which is hereby incorporated by reference) discloses certain compounds isolated after cultivation of a microorganism belonging to the genus Aspergillus, such as lovastatin. Also, U.S. Pat. No. 4,444,784 (the disclosure of which is hereby incorporated by reference) discloses synthetic derivatives of the aforementioned compounds, such as simvastatin. Also, U.S. Pat. No. 4,739,073 (the disclosure of which is incorporated by reference) discloses certain substituted indoles, such as fluvastatin. Also, U.S. Pat. No. 4,346,227 (the disclosure of which is incorporated by reference) discloses ML-236B derivatives, such as pravastatin. Also, EP 491226A (the disclosure of which is incorporated by reference) discloses certain pyridyidihydroxyheptenoic acids, such as cerivastatin. In addition, U.S. Pat. No. 5,273,995 (the disclosure of which is incorporated by reference) discloses certain 6-[2-(substituted-pyrrol-1-yl)alkyl]pyran-2-ones such as atorvastatin and any pharmaceutically acceptable form thereof (i.e. LIPITOR®). Additional HMG-CoA reductase inhibitors include rosuvastatin and pravastatin. Statins also include such compounds as rosuvastatin disclosed in U.S. RE37.314 E, pitivastatin disclosed in EP 304063 B1 and U.S. Pat. No. 5,011,930; mevastatin, disclosed in U.S. Pat. No. 3,983,140, which is incorporated herein by reference; velostatin, disclosed in U.S. Pat. No. 4,448,784 and U.S. Pat. No. 4,450,171, both of which are incorporated herein by reference; compactin, disclosed in U.S. Pat. No. 4,804,770, which is incorporated herein by reference; dalvastatin, disclosed in European Patent Application Publication No. 738510 A2; fluindostatin, disclosed in European Patent Application Publication No. 363934 A1; and dihydrocompactin, disclosed in U.S. Pat. No. 4,450,171, which is incorporated herein by reference.

Any PPAR modulator may be used in the combination aspect of this invention. The term PPAR modulator refers to compounds which modulate peroxisome proliferator activator receptor (PPAR) activity in mammals, particularly humans. Such modulation is readily determined by those skilled in the art according to standard assays known in the literature. It is believed that such compounds, by modulating the PPAR receptor, regulate transcription of key genes involved in lipid and glucose metabolism such as those in fatty acid oxidation and also those involved in high density lipoprotein (HDL) assembly (for example, apolipoprotein Al gene transcription), accordingly reducing whole body fat and increasing HDL cholesterol. By virtue of their activity, these compounds also reduce plasma levels of triglycerides, VLDL cholesterol, LDL cholesterol and their associated components such as apolipoprotein B in mammals, particularly humans, as well as increasing HDL cholesterol and apolipoprotein Al. Hence, these compounds are useful for the treatment and correction of the various dyslipidemias observed to be associated with the development and incidence of atherosclerosis and cardiovascular disease, including hypoalphalipoproteinemia and hypertriglyceridemia. A variety of these compounds are described and referenced below, however, others will be known to those skilled in the art. International Publication Nos. WO 02/064549 and 02/064130 and U.S. patent application Ser. No. 10/720,942, filed Nov. 24, 2003 and U.S. patent application 60/5521 14 filed Mar. 10, 2004 (the disclosures of which are hereby incorporated by reference) disclose certain compounds which are PPARA activators.

Any other PPAR modulator may be used in the combination aspect of this invention. In particular, modulators of PPARβ and/or PPARγ may be useful incombination with compounds of the present invention. An example PPAR inhibitor is described in US2003/02251 58 as {5-Methoxy-2-methyl-4-[4-(4-trifluoromethyl-benzyloxyj-benzylsulfanyl-phenoxyj-acetic acid.

Any MTP/Apo B (microsomal triglyceride transfer protein and or apolipoprotein B) secretion inhibitor may be used in the combination aspect of this invention. The term MTP/Apo B secretion inhibitor refers to compounds which inhibit the secretion of triglycerides, cholesteryl ester, and phospholipids. Such inhibition is readily determined by those skilled in the art according to standard assays (e.g., Wetterau, J. R. 1992; Science 258:999). A variety of these compounds are described and referenced below however other MTP/Apo B secretion inhibitors will be known to those skilled in the art, including imputapride (Bayer) and additional compounds such as those disclosed in WO 96/40640 and WO 98/23593, (two exemplary publications).

For example, the following MTP/Apo B secretion inhibitors are particularly useful:

4′-trifluoromethyl-biphenyl-2-carboxylic acid [2-(1H-1,2,4,]triazol-3-ylmethyl)-1,2,3,4-tetrahydro-isoquinolin-6-yl]-amide;

4′-trifluoromethylbiphenyl-2-carboxylic acid [2-(2-acetylamino-ethyl)-1,2,3,4-tetrahydro-isoquinolin-6-yl]-amide;

(2-{6-[(4′-trifluoromethyl-biphenyl-2-carbonyl)-amino]-3,4-dihydro-1H-isoquinolin-2-yl}-ethyl)-carbamic acid methyl ester;

4′-trifluoromethyl-biphenyl-2-carboxylic acid [2-(1H-imidazol-2-ylmethyl)-1,2,3,4-tetrahydro-isoquinolin-6-yl]-amide;

4′-trifluoromethyl-biphenyl-2-carboxylic acid [2-(2,2-diphenyl-ethyl)-1,2,3,4-tetrahydro-isoquinolin-6-yl]-amide;

4′-trifluoromethyl-biphenyl-2-carboxylic acid [2-(2-ethoxy-ethyl)-1,2,3,4-tetrahydro-isoquinolin-6-yl]-amide;

(S)-N-{2-[benzyl(methyl)amino]-2-oxo-1-phenylethyl}-1-methyl-5-t4′-trifluoromethyl)[1,1′-biphenyl]-2-carboxamido]-1H-indole-2-carboxamide;

(S)-2-t(4′-Trifluoromethyl-biphenyl-2-carbonyl)-amino]-quinoline-6-carboxylicacid (pentylcarbamoyi-phenyl-methyl)-amide;

1H-indole-2-carboxamide,1-methyl-N-[(1S)-2-[methyl(phenylmethyl)amino]-2-oxo-1-phenylethyl]-5-[[t4′-(trifluoromethyl)t1,1′-biphenyl]-2-y!]carbonyl]amino]; and

N-[(1S)-2-(benzylmethylamino)-2-oxo-1-phenylethyl]-1-methyl-5-[[t4′-(trifluoromethyl)biphenyl-2-yl]carbonyl]amino]-1H-indole-2-carboxamide.

Any HMG-CoA synthase inhibitor may be used in the combination aspect of this invention. The term HMG-CoA synthase inhibitor refers to compounds which inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by those skilled in the art according to standard assays (Meth Enzymol. 1975; 35:155-160: Meth. Enzymol. 1985; 110:19-26 and references cited therein). A variety of these compounds are described and referenced below, however other HMG-CoA synthase inhibitors will be known to those skilled in the art. U.S. Pat. No. 5,120,729 (the disclosure of which is hereby incorporated by reference) discloses certain beta-lactam derivatives. U.S. Pat. No. 5,064,856 (the disclosure of which is hereby incorporated by reference) discloses certain spiro-lactone derivatives prepared by culturing a microorganism (MF5253). U.S. Pat. No. 4,847,271 (the disclosure of which is hereby incorporated by reference) discloses certain oxetane compounds such as 11-(3-hydroxymethyl-4-oxo-2-oxetayl)-3,5,7-trimethyl-2,4-undeca-dienoic acid derivatives.

Any compound that decreases HMG-CoA reductase gene expression may be used in the combination aspect of this invention. These agents may be HMG-CoA reductase transcription inhibitors that block the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such compounds may cause this effect by decreasing levels of SREBP (sterol receptor binding protein) by inhibiting the activity of site-1 protease (S1 P) or agonizing the oxzgenal receptor or SCAP. Such regulation is readily determined by those skilled in the art according to standard assays (Meth. Enzymol. 1985; 110:9-19). Several compounds are described and referenced below, however other inhibitors of HMG-CoA reductase gene expression will be known to those skilled in the art. U.S. Pat. No. 5,041,432 (the disclosure of which is incorporated by reference) discloses certain 15-substituted lanosterol derivatives. Other oxygenated sterols that suppress synthesis of HMG-CoA reductase are discussed by E. I. Mercer (Prog. Lip. Res. 1993;32:357-416).

Any compound having activity as a CETP inhibitor can serve as the second compound in the combination therapy aspect of the present invention. The term CETP inhibitor refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art, for example, those disclosed in commonly assigned U.S. Pat. No. 6,140,343 and commonly assigned U.S. Pat. No. 6,197,786. CETP inhibitors disclosed in these patents include compounds, such as [2R.4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, which is also known as torcetrapib. CETP inhibitors are also described in U.S. Pat. No. 6,723,752, which includes a number of CETP inhibitors including (2R)-3-[3-(4-Chloro-3-ethyl-phenoxy)-phenyl]-[[3-(1,1,2,2-tetrafluoro-ethoxy)-phenyl)]-methyl]-amino}-1,1,1-trifluoro-2-propanol. Moreover, CETP inhibitors included herein are also described in U.S. patent application Ser. No. 10/807,838 filed Mar. 23, 2004. U.S. Pat. No. 5,512,548 discloses certain polypeptide derivatives having activity as CETP inhibitors, while certain CETP-inhibitory rosenonolactone derivatives and phosphate-containing analogs of cholesteryl ester are disclosed in J. Antibiot, 49(8): 815-816 (1996), and Bioorg. Med. Chem. Lett.; 6:1951-1954 (1996), respectively.

Any squalene synthetase inhibitor may be used in the combination aspect of this invention. The term squalene synthetase inhibitor refers to compounds which inhibit the condensation of 2 molecules of farnesylpyrophosphate to form squalene, catalyzed by the enzyme squalene synthetase. Such inhibition is readily determined by those skilled in the art according to standard assays (Meth. Enzymol. 1969; 15: 393-454 and Meth. Enzymol. 1985; 110:359-373 and references contained therein). A variety of these compounds are described in and referenced below however other squalene synthetase inhibitors will be known to those skilled in the art. U.S. Pat. No. 5,026,554 (the disclosure of which is incorporated by reference) discloses fermentation products of the microorganism MF5465 (ATCC 74011) including zaragozic acid. A summary of other patented squalene synthetase inhibitors has been compiled (Curr. Op. Ther. Patents (1993) 861-4).

Any squalene epoxidase inhibitor may be used in the combination aspect of this invention. The term squalene epoxidase inhibitor refers to compounds which inhibit the bioconversion of squalene and molecular oxygen into squalene-2,3-epoxide, catalyzed by the enzyme squalene epoxidase. Such inhibition is readily determined by those skilled in the art according to standard assays (Biochim. Biophys. Acta 1984; 794:466-471). A variety of these compounds are described and referenced below, however other squalene epoxidase inhibitors will be known to those skilled in the art. U.S. Pat. Nos. 5,011,859 and 5,064,864 (the disclosures of which are incorporated by reference) disclose certain fluoro analogs of squalene. EP publication 395,768 A (the disclosure of which is incorporated by reference) discloses certain substituted allylamine derivatives. PCT publication WO 9312069 A (the disclosure of which is hereby incorporated by reference) discloses certain amino alcohol derivatives. U.S. Pat. No. 5,051 ,534 (the disclosure of which is hereby incorporated by reference) discloses certain cydopropyloxy-squalene derivatives.

Any squalene cydase inhibitor may be used as the second component in the combination aspect of this invention. The term squalene cydase inhibitor refers to compounds which inhibit the bioconversion of squalene-2,3-epoxide to lanosterol, catalyzed by the enzyme squalene cydase. Such inhibition is readily determined by those skilled in the art according to standard assays (FEBS Lett. 1989;244:347-350.). In addition, the compounds described and referenced below are squalene cydase inhibitors, however other squalene cyclase inhibitors will also be known to those skilled in the art. PCT publication WO9410150 (the disclosure of which is hereby incorporated by reference) discloses certain 1,2,3,5,6,7,8,8a-octahydro-5,5,8(beta)-trimethyl-6-isoquinolineamine derivatives, such as N-trifluoroacetyl-1,2,3,5,6,7,8,8a-octahydro-2-allyl-5,5,8(beta)-trimethyl-6(beta)-isoquinolineamine. French patent publication 2697250 (the disclosure of which is hereby incorporated by reference) discloses certain beta, beta-dimethyl-4-piperidine ethanol derivatives such as 1-(1,5,9-trimethyldecyl)-beta,beta-dimethyl-4-piperidineethanol

Any combined squalene epoxidase/squalene cydase inhibitor may be used as the second component in the combination aspect of this invention. The term combined squalene epoxidase/squalene cydase inhibitor refers to compounds that inhibit the bioconversion of squalene to lanosterol via a squalene-2,3-epoxide intermediate. In some assays it is not possible to distinguish between squalene epoxidase inhibitors and squalene cyclase inhibitors, however, these assays are recognized by those skilled in the art. Thus, inhibition by combined squalene epoxidase/squalene cydase inhibitors is readily determined by those skilled in art according to the aforementioned standard assays for squalene cydase or squalene epoxidase inhibitors. A variety of these compounds are described and referenced below, however other squalene epoxidase/squalene cydase inhibitors will be known to those skilled in the art. U.S. Pat. Nos. 5,084,461 and 5,278,171 (the disclosures of which are incorporated by reference) disclose certain azadecalin derivatives. EP publication 468,434 (the disclosure of which is incorporated by reference) discloses certain piperidyl ether and thio-ether derivatives such as 2-(1-piperidyl)pentyl isopentyl sulfoxide and 2-(1-piperidyl)ethyl ethyl sulfide. PCT publication WO 9401404 (the disclosure of which is hereby incorporated by reference) discloses certain acyl-piperidines such as 1-(1-oxopentyl-5-phenylthio)-4-(2-hydroxy-1-methyl)-ethyl)piperidine. U.S. Pat. No. 5,102,915 (the disclosure of which is hereby incorporated by reference) discloses certain cydopropyloxy-squalene derivatives.

The compounds of the present invention can also be administered in combination with naturally occurring compounds that act to lower plasma cholesterol levels. These naturally occurring compounds are commonly called nutraceuticals and include, for example, garlic extract and niacin. A slow-release form of niacin is available and is known as Niaspan. Niacin may also be combined with other therapeutic agents such as lovastatin, or another is an HMG-CoA reductase inhibitor. This combination therapy with lovastatin is known as ADVICOR™ (Kos Pharmaceuticals Inc.).

Any cholesterol absorption inhibitor can be used as an additional in the combination aspect of the present invention. The term cholesterol absorption inhibition refers to the ability of a compound to prevent cholesterol contained within the lumen of the intestine from entering into the intestinal cells and/or passing from within the intestinal cells into the lymph system and/or into the blood stream. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480. An example of a recently approved cholesterol absorption inhibitor is ZETIA™ (ezetimibe) (Schering-Plough/Merck).

Any ACAT inhibitor may be used in the combination therapy aspect of the present invention. The term ACAT inhibitor refers to compounds that inhibit the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research., 24:1 127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity. Examples of ACAT inhibitors include compounds such as Avasimibe (Pfizer), CS-505 (Sankyo) and Eflucimibe (Eli Lilly and Pierre Fabre).

A lipase inhibitor may be used in the combination therapy aspect of the present invention. A lipase inhibitor is a compound that inhibits the metabolic cleavage of dietary triglycerides or plasma phospholipids into free fatty acids and the corresponding glycerides (e.g. EL, HL, etc.). Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a glyceride and fatty acid. In the intestine, the resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol. 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C K. Abrams, et al., Gastroenterology, 92,125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art. Preferred lipase inhibitors are those inhibitors that are selected from the group consisting of lipstatin, tetrahydrolipstatin (orlistat), valilactone, esterastin, ebelactone A, and ebelactone B. The compound tetrahydrolipstatin is especially preferred. The lipase inhibitor, N-3-trifluoromethylphenyl-N′-3-chloro-4′-trifluoromethylphenylurea, and the various urea derivatives related thereto, are disclosed in U.S. Pat. No. 4,405,644. The lipase inhibitor, esteracin, is disclosed in U.S. Pat. Nos. 4,189,438 and 4,242,453. The lipase inhibitor, cyclo-O,O′-[(1,6-hexanediyl-bis-(iminocarbonyl)]dioxime, and the various bis(iminocarbonyl)dioximes related thereto may be prepared as described in Petersen et al., Liebig's Annalen, 562, 205-229 (1949).

A variety of pancreatic lipase inhibitors are described herein below. The pancreatic lipase inhibitors lipstatin, (2S, 3S, 5S, 7Z, 10Z)-5-[(S)-2-formamido-4-methyl-valeryloxy]-2-hexyl-3-hydroxy-7,10-hexadecanoic acid lactone, and tetrahydrolipstatin (orlistat), (2S, 3S, 5S)-5-[(S)-2-formamido-4-methyl-valeryloxy]-2-hexyl-3-hydroxy-hexadecanoic 1,3 acid lactone, and the variously substituted N-formylleucine derivatives and stereoisomers thereof, are disclosed in U.S. Pat. No. 4,598,089. For example, tetrahydrolipstatin is prepared as described in, e.g., U.S. Pat. Nos. 5,274,143; 5,420,305; 5,540,917; and 5,643,874. The pancreatic lipase inhibitor, FL-386, 1-[4-(2-methylpropyl)cyclohexyl]-2-[(phenylsulfonyl)oxy]-ethanone, and the variously substituted sulfonate derivatives related thereto, are disclosed in U.S. Pat. No. 4,452,813. The pancreatic lipase inhibitor, WAY-121898, 4-phenoxyphenyl-4-methylpiperidin-1-yl-carboxylate, and the various carbamate esters and pharmaceutically acceptable salts related thereto, are disclosed in U.S. Pat. Nos. 5,512,565; 5,391,571 and 5,602,151. The pancreatic lipase inhibitor, valilactone, and a process for the preparation thereof by the microbial cultivation of Actinomycetes strain MG147-CF2, are disclosed in Kitahara, et al., J. Antibiotics, 40 (11), 1647-1650 (1987). The pancreatic lipase inhibitors, ebelactone A and ebelactone B, and a process for the preparation thereof by the microbial cultivation of Actinomycetes strain MG7-G1, are disclosed in Umezawa, etal., J. Antibiotics, 33, 1594-1596 (1980). The use of ebelactones A and B in the suppression of monoglyceride formation is disclosed in Japanese Kokai 08-143457, published Jun. 4, 1996.

Other compounds that are marketed for hyperlipidemia, including hypercholesterolemia and which are intended to help prevent or treat atherosclerosis include bile acid sequestrants, such as Welchol®, Colestid®, LoCholest® and Questran®; and fibric acid derivatives, such as Atromid®, Lopid® and Tricor®.

Diabetes can be treated by administering to a patient having diabetes (especially Type II), insulin resistance, impaired glucose tolerance, metabolic syndrome, or the like, or any of the diabetic complications such as neuropathy, nephropathy, retinopathy or cataracts, a therapeutically effective amount d a compound of the present invention in combination with other agents (e.g., insulin) that can be used to treat diabetes. This includes the classes of anti-diabetic agents (and specific agents) described herein.

Any glycogen phosphorylase inhibitor can be used as the second agent in combination with a compound of the present invention. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays (e.g., J. Med. Chem. 41 (1998) 2934-2938). A variety of glycogen phosphorylase inhibitors are known to those skilled in the art including those described in WO 96/39384 and WO 96/39385.

Any aldose reductase inhibitor can be used in combination with a compound of the present invention. The term aldose reductase inhibitor refers to compounds that inhibit the bioconversion of glucose to sorbitol, which is catalyzed by the enzyme aldose reductase. Aldose reductase inhibition is readily determined by those skilled in the art according to standard assays (e.g., J. Malone, Diabetes, 29:861-864 (1980). “Red Cell Sorbitol, an Indicator of Diabetic Control”). A variety of aldose reductase inhibitors are known to those skilled in the art.

Any sorbitol dehydrogenase inhibitor can be used in combination with a compound of the present invention. The term sorbitol dehydrogenase inhibitor refers to compounds that inhibit the bioconversion of sorbitol to fructose which is catalyzed by the enzyme sorbitol dehydrogenase. Such sorbitol dehydrogenase inhibitor activity is readily determined by those skilled in the art according to standard assays (e.g., Analyt. Biochem (2000) 280: 329-331). A variety of sorbitol dehydrogenase inhibitors are known, for example, U.S. Pat. Nos. 5,728,704 and 5,866,578 disclose compounds and a method for treating or preventing diabetic complications by inhibiting the enzyme sorbitol dehydrogenase.

Any glucosidase inhibitor can be used in combination with a compound of the present invention. A glucosidase inhibitor inhibits the enzymatic hydrolysis of complex carbohydrates by glycoside hydrolases, for example amylase or maltase, into bioavailable simple sugars, for example, glucose. The rapid metabolic action of glucosidases, particularly following the intake of high levels of carbohydrates, results in a state of alimentary hyperglycemia which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known chyme remaining in the stomach promotes the production of gastric juice, which initiates orfavors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969) 8:4214).

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethyl)-D-epi-inositol, and the various N-substituted pseudo-aminosugars related thereto, are disclosed in U.S. Pat. No. 4,701 ,559. The glucosidase inhibitor, miglitol, (2R,3R,4R,5S)-1-(2-hydroxyethyl)-2-(hydroxymethyl)-3,4,5-piperidinetriol and the various 3,4,5-trihydroxypiperidines related thereto, are disclosed in U.S. Pat. No. 4,639,436. The glucosidase inhibitor, emiglitate, ethyl p-[2-[(2R,3fi,4R,5S)-3,4,5-trihydroxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-0-β-D-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-t(2R,3R,4R,5S)-3,4,5-trihydroxy-2-(hydroxymethyl)piperidino]-α-D-glucopyranoside sesquihydrate, the deoxy-nojirimycin derivatives related thereto, the various pharmaceutically acceptable salts thereof and synthetic methods for the preparation thereof, are disclosed in U.S. Pat. Nos. 5,1 57,1 16 and 5,504,078. The glycosidase inhibitor, salbostatin and the various pseudosaccharides related thereto, are disclosed in U.S. Pat. No. 5,091,524.

A variety of amylase inhibitors are known to one of ordinary skill in the art. The amylase inhibitor, tendamistat and the various cyclic peptides related thereto, are disclosed in U.S. Pat. No. 4,451,455. The amylase inhibitor Al-3688 and the various cyclic polypeptides related thereto are disclosed in U.S. Pat. No. 4,623,714. The amylase inhibitor, trestatin, consisting of a mixture of trestatin A₁ trestatin B and trestatin C and the various trehalose-containing aminosugars related thereto are disclosed in U.S. Pat. No. 4,273,765.

Additional anti-diabetic compounds, which can be used as the second agent in combination with a compound of the present invention, include, for example, the following: biguanides (e.g., metformin), insulin secretagogues (e.g., sulfonylureas and glinides), glitazones, non-glitazone PPARy agonists, PPARγ agonists, inhibitors of DPP-IV, inhibitors of PDE5, inhibitors of GSK-3, glucagon antagonists, inhibitors of f-1,6-BPase(Metabasis/Sankyo), GLP-1/analogs (AC 2993, also known as exendin-4), insulin and insulin mimetics (Merck natural products). Other examples would include PKC-β inhibitors and AGE breakers.

The compounds of the present invention can be used in combination with anti-obesity agents. Any anti-obesity agent can be used as the second agent in such combinations and examples are provided herein. Such anti-obesity activity is readily determined by those skilled in the art according to standard assays known in the art.

Suitable anti-obesity agents include phenylpropanolamine, ephedrine, pseudoephedrine, phentermine, β3 adrenergic receptor agonists, apolipoprotein-B secretion/microsomal triglyceride transfer protein (apo-B/MTP) inhibitors, MCR4 agonists, cholecystokinin-A (CCK-A) agonists, monoamine reuptake inhibitors (e.g., sibutramine), sympathomimetic agents, serotoninergic agents, cannabinoid receptor (CB-1) antagonists (e.g., rimonabant described in U.S. Pat. No. 5,624,941 (SR-141.716A), purine compounds, such as those described in US Patent Publication No. 2004/0092520; pyrazolo[1,5-a][1,3,5]triazine compounds, such as those described in U.S. Non-Provisional patent application Ser. No. 10/763,105 filed on Jan. 21, 2004; and bicyclic pyrazolyl and imidazolyl compounds, such as those described in U.S. Provisional Application No. 60/518280 filed on Nov. 7, 2003), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelin receptor antagonists, histamine 3 receptor antagonists or inverse agonists, neuromedin U receptor agonists, and the like.

Any thyromimetic can be used as the second agent in combination with a compound of the present invention. Such thyromimetic activity is readily determined by those skilled in the art according to standard assays (e.g., Atherosclerosis (1996) 126: 53-63). A variety of thyromimetic agents are known to those skilled in the art, for example those disclosed in U.S. Pat. Nos. 4,766,121; 4,826,876; 4,910,305; 5,061,798; 5,284,971; 5,401,772; 5,654,468; and 5,569,674. Other antiobesity agents include sibutramine which can be prepared as described in U.S. Pat. No. 4,929,629. and bromocriptine which can be prepared as described in U.S. Pat. Nos. 3,752,814 and 3,752,888.

The compounds of the present invention can also be used in combination with other antihypertensive agents. Any anti-hypertensive agent can be used as the second agent in such combinations and examples are provided herein. Such antihypertensive activity is readily determined by those skilled in the art according to standard assays (e.g., blood pressure measurements).

Examples of presently marketed products containing antihypertensive agents include calcium channel blockers, such as Cardizem®, Adalat®, Calan®, Cardene®, Covera®, Dilacor®, DynaCirc®, Procardia XL®, Sular®, Tiazac®, Vascor®, Verelan®, Isoptin®, Nimotop®, Norvasc®, and Plendil®; angiotensin converting enzyme (ACE) inhibitors, such as Accupril®, Altace®, Captopril®, Lotensin®, Mavik®, Monopril®, Prinivil®, Univasc®, Vasotec® and Zestril®.

Amlodipine and related dihydropyridine compounds are disclosed in U.S. Pat. No. 4,572,909, which is incorporated herein by reference, as potent anti-ischemic and antihypertensive agents. U.S. Pat. No. 4,879,303, which is incorporated herein by reference, discloses amlodipine benzenesulfonate salt (also termed amlodipine besylate). Amlodipine and amlodipine besylate are potent and long lasting calcium channel blockers. As such, amlodipine, amlodipine besylate, amlodipine maleate and other pharmaceutically acceptable acid addition salts of amlodipine have utility as antihypertensive agents and as antiischemic agents. Amlodipine besylate is currently sold as Norvasc®. Amlodipine has the formula

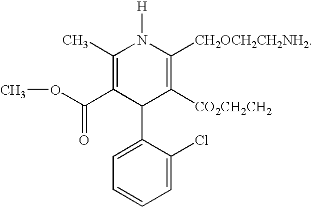

Calcium channel blockers which are within the scope of this invention include, but are not limited to: bepridil, which may be prepared as disclosed in U.S. Pat. No.3,962, 238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, which may be prepared as disclosed in U.S. Pat. No. 4,808,605; prenylamine, which may be prepared as disclosed in U.S. Pat. No. 3,152,173; semotiadil, which may be prepared as disclosed in U.S. Pat. No. 4,786,635; terodiline, which may be prepared as disclosed in U.S. Pat. No. 3,371,014; verapamil, which may be prepared as disclosed in U.S. Pat. No. 3,261,859; aranipine, which may be prepared as disclosed in U.S. Pat. No. 4,572,909; bamidipine, which may be prepared as disclosed in U.S. Pat. No. 4,220,649; benidipine, which may be prepared as disclosed in European Patent Application Publication No. 106,275; cilnidipine, which may be prepared as disclosed in U.S. Pat. No. 4,672,068; efonidipine, which may be prepared as disclosed in U.S. Pat. No. 4,885,284; elgodipine, which may be prepared as disclosed in U.S. Pat. No. 4,952,592; felodipine, which may be prepared as disclosed in U.S. Pat. No. 4,264,611; isradipine, which may be prepared as disclosed in U.S. Pat. No. 4,466,972; laddipine, which may be prepared as disdosed in U.S. Pat. No. 4,801,599; lercanidipine, which may be prepared as disclosed in U.S. Pat. No. 4,705,797; manidipine, which may be prepared as disclosed in U.S. Pat. No. 4,892,875; nicardipine, which may be prepared as disclosed in U.S. Pat. No. 3,985,758; nifedipine, which may be prepared as disclosed in U.S. Pat. No. 3,485,847; nilvadipine, which may be prepared as disclosed in U.S. Pat. No. 4,338,322; nimodipine, which may be prepared as disclosed in U.S. Pat. No. 3,799,934; nisoldipine, which may be prepared as disclosed in U.S. Pat. No. 4,154,839; nitrendipine, which may be prepared as disclosed in U.S. Pat. No. 3,799,934; cinnarizine, which may be prepared as disclosed in U.S. Pat. No. 2,882,271; flunarizine, which may be prepared as disclosed in U.S. Pat. No. 3,773,939; lidoflazine, which may be prepared as disclosed in U.S. Pat. No. 3,267,104; lomerizine, which may be prepared as disclosed in U.S. Pat. No. 4,663,325; bencyclane, which may be prepared as disclosed in Hungarian Patent No. 151,865; etafenone, which may be prepared as disclosed in German Patent No. 1,265,758; and perhexiline, which may be prepared as disclosed in British Patent No. 1,025,578. The disclosures of all such U.S. Patents are incorporated herein by reference.

Angiotensin Converting Enzyme Inhibitors (ACE-Inhibitors) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveitopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361 . The disclosures of all such U.S. patents are incorporated herein by reference.

Angiotensin-II receptor antagonists (A-II antagonists) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; and valsartan, which may be prepared as disclosed in U.S. Pat. No. 5,399,578. The disclosures of all such U.S. patents are incorporated herein by reference.

Beta-adrenergic receptor blockers (beta- or β-blockers) which are within the scope of this invention include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. No. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may be prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071; buprandolol, which may be prepared as disclosed in U.S. Pat. No. 3,309,406; butiridine hydrochloride, which may be prepared as disclosed in French Patent No. 1,390,056; butofilolol, which may be prepared as disclosed in U.S. Pat. No. 4,252,825; carazolol, which may be prepared as disclosed in German Patent No. 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; cloranolol, which may be prepared as disclosed in German Patent No. 2,213,044; dilevalol, which may be prepared as disclosed in Clifton et al., Journal of Medicinal Chemistry, 1982, 25, 670; epanolol, which may be prepared as disclosed in European Patent Publication Application No. 41,491; indenolol, which may be prepared as disclosed in U.S. Pat. No. 4,045,482; labetalol, which may be prepared as disclosed in U.S. Pat. No. 4,012,444; levobunolol, which may be prepared as disclosed in U.S. Pat. No. 4,463,176; mepindolol, which may be prepared as disclosed in Seeman et al., Helv. Chim. Acta, 1971, 54, 241; metipranolol, which may be prepared as disclosed in Czechoslovakian Patent Application No. 128,471; metoprolol, which may be prepared as disclosed in U.S. Pat. No. 3,873,600; moprolol, which may be prepared as disclosed in U.S. Pat. No. 3,501 ,7691; nadolol, which may be prepared as disclosed in U.S. Pat. No. 3,935, 267; nadoxolol, which may be prepared as disclosed in U.S. Pat. No. 3,819,702; nebivalol, which may be prepared as disclosed in U.S. Pat. No. 4,654,362; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,357; propranolol, which may be prepared as disclosed in U.S. Pat. Nos. 3,337,628 and 3,520,919; sotalol, which may be prepared as disclosed in Uloth et al., Journal of Medicinal Chemistry, 1966, 9, 88; sufinalol, which may be prepared as disclosed in German Patent No. 2,728,641; talindol, which may be prepared as disclosed in U.S. Pat. Nos. 3,935,259 and 4,038,313; tertatolol, which may be prepared as disclosed in U.S. Pat. No. 3,960,891; tilisolol, which may be prepared as disclosed in U.S. Pat. No. 4,129,565; timolol, which may be prepared as disclosed in U.S. Pat. No. 3,655,663; toliprolol, which may be prepared as disclosed in U.S. Pat. No. 3,432,545; and xibenolol, which may be prepared as disclosed in U.S. Pat. No. 4,018,824. The disclosures of all such U.S. patents are incorporated herein by reference.

Alpha-adrenergic receptor blockers (alpha- or α-blockers) which are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,51 1,836; tamsulosin, which may be prepared as disclosed in U.S. Pat. No. 4,703,063; tolazoline, which may be prepared as disclosed in U.S. Pat. No. 2,161,938; trimazosin, which may be prepared as disclosed in U.S. Pat. No. 3,669,968; and yohimbine, which may be isolated from natural sources according to methods well known to those skilled in the art. The disclosures of all such U.S. patents are incorporated herein by reference.

The term “vasodilator,” where used herein, is meant to include cerebral vasodilators, coronary vasodilators and peripheral vasodilators. Cerebral vasodilators within the scope of this invention include, but are not limited to: bencyclane; cinnarizine; citicoline, which may be isolated from natural sources as disclosed in Kennedy et al., Journal of the American Chemical Society, 1955, 77, 250 or synthesized as disclosed in Kennedy, Journal of Biological Chemistry, 1956, 222, 185; cyclandelate, which may be prepared as disclosed in U.S. Pat. No. 3,663,597; ciclonicate, which may be prepared as disclosed in German Patent No. 1,910,481; diisopropylamine dichloroacetate, which may be prepared as disclosed in British Patent No. 862,248; ebumamonine, which may be prepared as disclosed in Hermann et al., Journal of the American Chemical Society, 1979, 101, 1540; fasudil, which may be prepared as disclosed in U.S. Pat. No. 4,678,783; fenoxedil, which may be prepared as disclosed in U.S. Pat. No. 3,818,021; flunarizine, which may be prepared as disclosed in U.S. Pat. No. 3,773,939; ibudilast, which may be prepared as disclosed in U.S. Pat. No. 3,850,941; ifenprodil, which may be prepared as disclosed in U.S. Pat. No. 3,509,164; lomerizine, which may be prepared as disclosed in U.S. Pat. No. 4,663,325; nafronyl, which may be prepared as disclosed in U.S. Pat. No. 3,334,096; nicametate, which may be prepared as disclosed in Blicke et al., Journal of the American Chemical Society, 1942, 64, 1722; nicergoline, which may be prepared as disclosed above; nimodipine, which may be prepared as disclosed in U.S. Pat. No. 3,799,934; papaverine, which may be prepared as reviewed in Goldberg, Chem. Prod. Chem. News, 1954, 17 371; pentifylline, which may be prepared as disclosed in German Patent No. 860,217; tinofedrine, which may be prepared as disclosed in U.S. Pat. No. 3,563,997; vincamine, which may be prepared as disclosed in U.S. Pat. No. 3,770,724; vinpocetine, which may be prepared as disclosed in U.S. Pat. No. 4,035,750; and viquidil, which may be prepared as disclosed in U.S. Pat. No. 2,500,444. The disclosures of all such U.S. patents are incorporated herein by reference.

Coronary vasodilators within the scope of this invention include, but are not limited to: amotriphene, which may be prepared as disclosed in U.S. Pat. No. 3,010,965; bendazol, which may be prepared as disclosed in J. Chem. Soc. 1958, 2426; benfurodil hemisuccinate, which may be prepared as disclosed in U.S. Pat. No. 3,355,463; benziodarone, which may be prepared as disclosed in U.S. Pat. No. 3,012,042; chloracizine, which may be prepared as disclosed in British Patent No. 740,932; chromonar, which may be prepared as disclosed in U.S. Pat. No. 3,282,938; clobenfural, which may be prepared as disclosed in British Patent No. 1,160,925; clonitrate, which may be prepared from propanediol according to methods well known to those skilled in the art, e.g., see Annalen, 1870, 155, 165; cloricromen, which may be prepared as disclosed in U.S. Pat. No. 4,452,811; dilazep, which may be prepared as disclosed in U.S. Pat. No. 3,532,685; dipyridamole, which may be prepared as disclosed in British Patent No. 807,826; droprenilamine, which may be prepared as disclosed in German Patent No. 2,521,113; efloxate, which may be prepared as disclosed in British Patent Nos. 803,372 and 824,547; erythrityl tetranitrate, which may be prepared by nitration of erythritol according to methods well-known to those skilled in the art; etafenone, which may be prepared as disclosed in German Patent No. 1,265,758; fendiline, which may be prepared as disclosed in U.S. Pat. No. 3,262,977; floredil, which may be prepared as disclosed in German Patent No. 2,020,464; ganglefene, which may be prepared as disclosed in U.S.S.R. Patent No. 115,905; hexestrol, which may be prepared as disclosed in U.S. Pat. No. 2,357,985; hexobendine, which may be prepared as disclosed in U.S. Pat. No. 3,267,103; itramin tosylate, which may be prepared as disclosed in Swedish Patent No. 168,308; khellin, which may be prepared as disclosed in Baxter et al., Journal of the Chemical Society, 1949, S 30; lidoflazine, which may be prepared as disclosed in U.S. Pat. No. 3,267,104; mannitol hexanitrate, which may be prepared by the nitration of mannitol according to methods well-known to those skilled in the art; medibazine, which may be prepared as disclosed in U.S. Pat. No. 3,119,826; nitroglycerin; pentaerythritol tetranitrate, which may be prepared by the nitration of pentaerythritol according to methods well-known to those skilled in the art; pentrinitrol, which may be prepared as disclosed in German Patent No. 638,422-3; perhexilline, which may be prepared as disclosed above; pimefylline, which may be prepared as disclosed in U.S. Pat. No. 3,350,400; prenylamine, which may be prepared as disclosed in U.S. Pat. No. 3,152,173; propatyl nitrate, which may be prepared as disclosed in French Patent No. 1,103,113; trapidil, which may be prepared as disclosed in East German Patent No. 55,956; tricromyl, which may be prepared as disclosed in U.S. Pat. No. 2,769,015; trimetazidine, which may be prepared as disclosed in U.S. Pat. No. 3,262,852; trolnitrate phosphate, which may be prepared by nitration of triethanolamine followed by precipitation with phosphoric acid according to methods well-known to those skilled in the art; visnadine, which may be prepared as disclosed in U.S. Pat. Nos. 2,816,118 and 2,980,699. The disclosures of all such U.S. patents are incorporated herein by reference.

Peripheral vasodilators within the scope of this invention include, but are not limited to: aluminum nicotinate, which may be prepared as disclosed in U.S. Pat. No. 2,970,082; bamethan, which may be prepared as disclosed in Corrigan et al., Journal of the American Chemical Society, 1945, 67,1894; bencyclane, which may be prepared as disclosed above; betahistine, which may be prepared as disclosed in Walter et al.; Journal of the American Chemical Society. 1941, 63, 2771; bradykinin, which may be prepared as disclosed in Hamburg et al., Arch. Biochem. Biophys., 1958, 76, 252; brovincamine, which may be prepared as disclosed in U.S. Pat. No. 4,146,643; bufeniode, which may be prepared as disclosed in U.S. Pat. No. 3,542,870; buflomedil, which may be prepared as disclosed in U.S. Pat. No. 3,895,030; butalamine, which may be prepared as disclosed in U.S. Pat. No. 3,338,899; cetiedil, which may be prepared as disclosed in French Patent Nos. 1,460,571; ciclonicate, which may be prepared as disclosed in German Patent No. 1,910,481; cinepazide, which may be prepared as disclosed in Belgian Patent No. 730,345; cinnarizine, which may be prepared as disclosed above; cyclandelate, which may be prepared as disclosed above; dilsopropylamine dichloroacetate, which may be prepared as disclosed above; eledoisin, which may be prepared as disclosed in British Patent No. 984,810; fenoxedil, which may be prepared as disclosed above; flunarizine, which may be prepared as disclosed above; hepronicate, which may be prepared as disclosed in U.S. Pat. No. 3,384,642; ifenprodil, which may be prepared as disclosed above; iloprost, which may be prepared as disclosed in U.S. Pat. No. 4,692,464; inositol niacinate, which may be prepared as disclosed in Badgett et al., Journal of the American Chemical Society, 1947, 69, 2907; isoxsuprine, which may be prepared as disclosed in U.S. Pat. No. 3,056,836; kallidin, which may be prepared as disclosed in Biochem. Biophys. Res. Commun., 1961, 6, 210; kallikrein, which may be prepared as disclosed in German Patent No. 1,102,973; moxisylyte, which may be prepared as disclosed in German Patent No. 905,738; nafronyl, which may be prepared as disclosed above; nicametate, which may be prepared as disclosed above; nicergoline, which may be prepared as disclosed above; nicofuranose, which may be prepared as disclosed in Swiss Patent No. 366,523; nylidrin, which may be prepared as disclosed in U.S. Pat. Nos. 2,661,372 and 2,661,373; pentifylline, which may be prepared as disclosed above; pentoxifylline, which may be prepared as disclosed in U.S. Pat. No. 3,422,107; piribedil, which may be prepared as disclosed in U.S. Pat. No. 3,299,067; prostaglandin Ei, which may be prepared by any of the methods referenced in the Merck Index, Twelfth Edition, Budaveri, Ed., New Jersey, 1996, p. 1353; suloctidil, which may be prepared as disclosed in German Patent No. 2,334,404; tolazoline, which may be prepared as disclosed in U.S. Pat. No. 2,161,938; and xanthinol niacinate, which may be prepared as disclosed in German Patent No. 1,102,750 or Korbonits et al., Acta. Pharm. Hung., 1968.38, 98. The disclosures of all such U.S. patents are incorporated herein by reference.

The term “diuretic,” within the scope of this invention, is meant to include diuretic benzothiadiazine derivatives, diuretic organomercurials, diuretic purines, diuretic steroids, diuretic sulfonamide derivatives, diuretic uracils and other diuretics such as amanozine, which may be prepared as disclosed in Austrian Patent No. 168,063; amiloride, which may be prepared as disdosed in Belgian Patent No. 639,386; arbutin, which may be prepared as disclosed in Tschitschibabin, Annalen, 1930, 479, 303; chlorazanil, which may be prepared as disclosed in Austrian Patent No. 168,063; ethacrynic acid, which may be prepared as disclosed in U.S. Pat. No. 3,255,241; etozolin, which may be prepared as disclosed in U.S. Pat. No. 3,072,653; hydracarbazine, which may be prepared as disclosed in British Patent No. 856,409; isosorbide, which may be prepared as disclosed in U.S. Pat. No. 3,160,641; mannitol; metochalcone, which may be prepared as disdosed in Freudenberg et al., Ber., 1957, 90. 957; muzolimine, which may be prepared as disclosed in U.S. Pat. No. 4,018,890; perhexiline, which may be prepared as disclosed above; ticrynafen, which may be prepared as disclosed in U.S. Pat. No. 3,758,506; triamterene which may be prepared as disdosed in U.S. Pat. No. 3,081 ,230; and urea. The disdosures of all such U.S. patents are incorporated herein by reference.

Diuretic benzothiadiazine derivatives within the scope of this invention include, but are not limited to: althiazide, which may be prepared as disclosed in British Patent No. 902,658; bendroflumethiazide, which may be prepared as disclosed in U.S. Pat. No. 3,265,573; benzthiazide, McManus et al., 136th Am. Soc. Meeting (Atlantic City, September 1959), Abstract of papers, pp 13-0; benzylhydrochlorothiazide, which may be prepared as disclosed in U.S. Pat. No. 3,108,097; buthiazide, which may be prepared as disclosed in British Patent Nos. 861 ,367 and 885,078; chlorothiazide, which may be prepared as disdosed in U.S. Pat. Nos. 2,809,194 and 2,937,169; chlorthalidone, which may be prepared as disdosed in U.S. Pat. No. 3,055,904; cyclopenthiazide, which may be prepared as disdosed in Belgian Patent No. 587,225; cyclothiazide, which may be prepared as disclosed in Whitehead et al., Journal of Organic Chemistry, 1961,26, 2814; epithiazide, which may be prepared as disclosed in U.S. Pat. No. 3,009,911; ethiazide, which may be prepared as disclosed in British Patent No. 861,367; fenquizone, which may be prepared as disclosed in U.S. Pat. No. 3,870,720; indapamide, which may be prepared as disdosed in U.S. Pat. No. 3,565,911; hydrochlorothiazide, which may be prepared as disdosed in U.S. Pat. No. 3,164,588; hydroflumethiazide, which may be prepared as disdosed in U.S. Pat. No. 3,254,076; methyclothiazide, which may be prepared as disclosed in Close et al., Journal of the American Chemical Society, 1960, 82, 1132; meticrane, which may be prepared as disclosed in French Patent Nos. M2790 and 1,365,504; metolazone, which may be prepared as disclosed in U.S. Pat. No. 3,360,518; paraflutizide, which may be prepared as disclosed in Belgian Patent No. 620,829; polythiazide, which may be prepared as disclosed in U.S. Pat. No. 3,009,911; quinethazone, which may be prepared as disclosed in U.S. Pat. No. 2,976,289; teclothiazide, which may be prepared as disclosed in Close et al., Journal of the American Chemical Society, 1960, 82, 1132; and trichlormethiazide, which may be prepared as dislcosed in deStevens et al., Experientia, 1960, 16, 113. The disdosures of all such U.S. patents are incorporated herein by reference.

Diuretic sulfonamide derivatives within the scope of this invention include, but are not limited to: acetazolamide, which may be prepared as disdosed in U.S. Pat. No. 2,980,679; ambuside, which may be prepared as disclosed in U.S. Pat. No. 3,188,329; azosemide, which may be prepared as disclosed in U.S. Pat. No. 3,665,002; bumetanide, which may be prepared as disclosed in U.S. Pat. No. 3,634,583; butazolamide, which may be prepared as disclosed in British Patent No. 769,757; chloraminophenamide, which may be prepared as disclosed in U.S. Pat. Nos. 2,809,194, 2,965,655 and 2,965,656; clofenamide, which may be prepared as disclosed in Olivier, Rec. Trav. Chim., 1918, 37, 307; clopamide, which may be prepared as disclosed in U.S. Pat. No. 3,459,756; clorexolone, which may be prepared as disclosed in U.S. Pat. No. 3,183,243; disulfamide, which may be prepared as disclosed in British Patent No. 851,287; ethoxolamide, which may be prepared as disclosed in British Patent No. 795,174; furosemide, which may be prepared as disclosed in U.S. Pat. No. 3,058,882; mefruside, which may be prepared as disclosed in U.S. Pat. No. 3,356,692; methazolamide, which may be prepared as disclosed in U.S. Pat. No. 2,783,241; piretanide, which may be prepared as disclosed in U.S. Pat. No. 4,010,273; torasemide, which may be prepared as disclosed in U.S. Pat. No. 4,018,929; tripamide, which may be prepared as disclosed in Japanese Patent No. 73 05,585; and xipamide, which may be prepared as disclosed in U.S. Pat. No. 3,567,777. The disclosures of all such U.S. patents are incorporated herein by reference.

Osteoporosis is a systemic skeletal disease, characterized by low bone mass and deterioration of bone tissue, with a consequent increase in bone fragility and susceptibility to fracture. In the U.S., the condition affects more than 25 million people and causes more than 1.3 million fractures each year, including 500,000 spine, 250,000 hip and 240,000 wrist fractures annually. Hip fractures are the most serious consequence of osteoporosis, with 5-20% of patients dying within one year, and over 50% of survivors being incapacitated.

The elderly are at greatest risk of osteoporosis, and the problem is therefore predicted to increase significantly with the aging of the population. Worldwide fracture incidence is forecasted to increase three-fold over the next 60 years, and one study has estimated that there will be 4.5 million hip fractures worldwide in 2050.

Women are at greater risk of osteoporosis than men. Women experience a sharp acceleration of bone loss during the five years following menopause. Other factors that increase the risk include smoking, alcohol abuse, a sedentary lifestyle and low calcium intake.

Those skilled in the art will recognize that anti-resorptive agents (for example progestins, polyphosphonates, bisphosphonate(s), estrogen agonists/antagonists, estrogen, estrogen/progestin combinations, Premarin®, estrone, estriol or 17α- or 17β-ethynyl estradiol) may be used in conjunction with the compounds of the present invention.

Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, flurogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol.

Preferred progestins are medroxyprogestrone, norethindrone and norethynodrel.

Exemplary bone resorption inhibiting polyphosphonates include polyphosphonates of the type disclosed in U.S. Pat. No. 3,683,080, the disclosure of which is incorporated herein by reference. Preferred polyphosphonates are geminal diphosphonates (also referred to as bis-phosphonates). Tiludronate disodium is an especially preferred polyphosphonate. Ibandronic acid is an especially preferred polyphosphonate. Alendronate and resindronate are especially preferred polyphosphonates. Zoledronic acid is an especially preferred polyphosphonate. Other preferred polyphosphonates are 6-amino-1 -hydroxy-hexylidene-bisphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid.N.N-dimethylamino methane diphosphonic acid, N(2-hydroxyethyl) amino methane diphosphonic acid, butane-4-amino-1-hydroxy-1,1-diphosphonic acid, pentane-5-amino-1-hydroxy-1,1-diphosphonic acid, hexane-6-amino-1-hydroxy-1,1-diphosphonic acid and pharmaceutically acceptable esters and salts thereof.

In particular, the compounds of this invention may be combined with a mammalian estrogen agonist/antagonist. Any estrogen agonist/antagonist may be used in the combination aspect of this invention. The term estrogen agonist/antagonist refers to compounds which bind with the estrogen receptor, inhibit bone turnover and/or prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue, and blocking the actions of estrogen in one or more tissues. Such activities are readily determined by those skilled in the art of standard assays including estrogen receptor binding assays, standard bone histomorphometric and densitometer methods, and Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below.

Another preferred estrogen agonist/antagonist is 3-(4-(1,2-diphenyl-but-1-enyl)-phenyl)-acrylic acid, which is disclosed in Willson et al., Endocrinology, 1997, 138, 3901-3911.

Another preferred estrogen agonist/antagonist is tamoxifen: (ethanamine,2-(-4-1,2-diphenyl-1-butenyl)phenoxy)-N,N-dimethyl, (Z)-2-, 2-hydroxy-1,2,3-propanetricarboxylate(1:1)) and related compounds which are disclosed in U.S. Pat. No. 4,536,516, the disclosure of which is incorporated herein by reference.

Another related compound is 4-hydroxy tamoxifen, which is disclosed in U.S. Pat. No. 4,623,660, the disclosure of which is incorporated herein by reference.

A preferred estrogen agonist/antagonist is raloxifene: (methanone, (6-hydroxy-2-(4-hydroxyphenyl)benzo[b]thien-3-yl)(4-(2-(1-piperidinyl)ethoxy)phenyl)-hydrochloride) which is disclosed in U.S. Pat. No. 4,418,068, the disclosure of which is incorporated herein by reference.

Another preferred estrogen agonist/antagonist is toremifene: (ethanamine, 2-(4-(4-chloro-1,2-diphenyl-1-butenyl)phenoxy)-N,N-dimethyl-, (Z)-, 2-hydroxy-1,2,3-propanetricarboxylate (1:1) which is disclosed in U.S. Pat. No. 4,996,225, the disclosure of which is incorporated herein by reference.

Another preferred estrogen agonist/antagonist is centchroman: 1-(2-((4-(-methoxy-2,2, dimethyl-3-phenyl-chroman-4-yl)-phenoxy)-ethyl)-pyrrolidine, which is disclosed in U.S. Pat. No. 3,822,287, the disclosure of which is incorporated herein by reference. Also preferred is levormeloxifene.

Another preferred estrogen agonist/antagonist is idoxifene: (E)-1-(2-(4-(1-(4-iodo-phenyl)-2-phenyl-but-1-enyl)-phenoxy)-ethyl)-pyrrolidinone, which is disclosed in U.S. Pat. No. 4,839,155, the disclosure of which is incorporated herein by reference.

Another preferred estrogen agonist/antagonist is 2-(4-methoxy-phenyl)-3-[4-(2-piperidin-1-yl-ethoxy)-phenoxy]-benzo[b]thiophen-6-ol which is disclosed in U.S. Pat. No. 5,488,058, the disclosure of which is incorporated herein by reference.

Another preferred estrogen agonist/antagonist is 6-(4-hydroxy-phenyl)-5-(4-(2-piperidin-1-yl-ethoxy)-benzyl)-naphthalen-2-ol, which is disclosed in U.S. Pat. No. 5,484,795, the disclosure of which is incorporated herein by reference.

Another preferred estrogen agonist/antagonist is (4-(2-(2-aza-bicyclo[2.2.1]hept-2-yl)-ethoxy)-phenyl)-(6-hydroxy-2-(4-hydroxy-phenyl)benzo[b]thiophen-3-yl)-methanone which is disclosed, along with methods of preparation, in PCT publication no. WO 95/10513 assigned to Pfizer Inc.

Other preferred estrogen agonist/antagonists include the compounds, TSE-424 (Wyeth-Ayerst Laboratories) and arazoxifene.

Other preferred estrogen agonist/antagonists include compounds as described in commonly assigned U.S. Pat. No. 5,552,412, the disclosure of which is incorporated herein by reference. Especially preferred compounds described therein are:

c/s-6-(4-fluoro-phenyl)-5-(4-(2-piperidin-1 -yl-ethoxyphenyl)-5,6,7,8-tetrahydro-naphthalene-2-ol;

(-)-c/s-6-phenyl-5-(4-(2-pyrrolidin-1-yl-ethoxy)-phenyl)-5,6,7,8-tetrahydro-naphthalene-2-ol (also known as lasofoxifene);

c/s-6-phenyl-5-(4-(2-pyrrolidin-1-yl-ethoxy)-phenyl)-5,6,7,8-tetrahydro-naphthalene-2-ol;

c/s-1-(6′-pyrrolodinoethoxy-3′-pyridyl)-2-phenyl-6-hydroxy-1,2,3,4-tetrahydronaphthalene;

1-(4′-pyrrolidinoethoxyphenyl)-2-(4″-fluorophenyl)-6-hydroxy-1,2,3,4-tetrahydroisoquinoline;

c/s-6-(4-hydroxyphenyl)-5-(4-(2-piperidin-1 -yl-ethoxy)-phenyl)-5,6,7,8-tetrahydro-naphthalene-2-ol; and

1-(4′-pyrrolidinolethoxyphenyl)-2-phenyl-6-hydroxy-1,2,3,4-tetrahydroisoquinoline.

Other estrogen agonist/antagonists are described in U.S. Pat. No. 4,133,814 (the disclosure of which is incorporated herein by reference). U.S. Pat. No. 4,1 33,814 discloses derivatives of 2-phenyl-3-aroyl-benzothiophene and 2-phenyl-3-aroylbenzothiophene-1-oxide.

Other anti-osteoporosis agents, which can be used as the second agent in combination with a compound of the present invention, include, for example, the following: parathyroid hormone (PTH) (a bone anabolic agent); parathyroid hormone (PTH) secretagogues (see, e.g., U.S. Pat. No. 6,132,774), particularly calcium receptor antagonists; calcitonin; and vitamin D and vitamin D analogs.

Any selective androgen receptor modulator (SARM) can be used in combination with a compound of the present invention. A selective androgen receptor modulator (SARM) is a compound that possesses androgenic activity and which exerts tissue-selective effects. SARM compounds can function as androgen receptor agonists, partial agonists, partial antagonists or antagonists. Examples of suitable SARMs include compounds such as cyproterone acetate, chlormadinone, flutamide, hydroxyflutamide, bicalutamide, nilutamide, spironolactone, 4-(trifluoromethyl)-2(1H)-pyrrolidino[3,2-g]quinoline derivatives, 1,2-dihydropyridino[5,6-g]quinoline derivatives and piperidino[3,2-g]quinolinone derivatives.

Cypterone, also known as (1b,2b)-6-chloro-1,2-dihydro-17-hydroxy-3′H-cyclopropa[1,2]pregna-1,4,6-triene-3,20-dione is disclosed in U.S. Pat. No. 3,234,093. Chlormadinone, also known as 17-(acetyloxy)-6-chloropregna-4,6-diene-3,20-dione, in its acetate form, acts as an anti-androgen and is disclosed in U.S. Pat. No. 3,485,852. Nilutamide, also known as 5,5-dimethyl-3-[4-nito-3-(trifluoromethyl)phenyl]-2,4-imidazolidinedione and by the trade name Nilandron® is disclosed in U.S. Pat. No. 4,097,578. Flutamide, also known as 2-methyl-N-[4-nitro-3-(trifluoromethyl)phenyl]propanamide and the trade name Eulexin® is disclosed in U.S. Pat. No. 3,847,988. Bicalutamide, also known as 4′-cyano-a′,a′,a′-trifluoro-3-(4-fluorophenylsulfonyl)-2-hydroxy-2-methylpropiono-m-toluidide and the trade name Casodex® is disclosed in EP-100172. The enantiomers of biclutamide are discussed by Tucker and Chesterton, J. Med. Chem. 1988, 31, 885-887. Hydroxyflutamide, a known androgen receptor antagonist in most tissues, has been suggested to function as a SARM for effects on IL-6 production by osteoblasts as disclosed in Hofbauer et al. J. Bone Miner. Res. 1999, 14, 1330-1337. Additional SARMs have been disclosed in U.S. Pat. No. 6,017,924; WO 01/16108, WO 01/16133, WO 01/16139, WO 02/00617, WO 02/16310, U.S. Patent Application Publication No. US 2002/0099096, U.S. Patent Application Publication No. US 2003/0022868, WO 03/011302 and WO 03/011824. All of the above refences are hereby incorporated by reference herein.

The starting materials and reagents for the above described compounds, are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Some of the compounds of this invention or intermediates in their synthesis have asymmetric carbon atoms and therefore are enantiomers or diastereomers. Diasteromeric mixtures can be separated into their individual diastereomers on the basis of their physical chemical differences by methods known. pre se, for example, by chromatography and/or fractional crystallization. Enantiomers can be separated by, for example, chiral HPLC methods or converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. Also, an enantiomeric mixture of the compounds or an intermediate in their synthesis which contain an acidic or basic moiety may be separated into their corresponding pure enantiomers by forming a diastereomic salt with an optically pure chiral base or acid (e.g., 1-phenyl-ethyl amine, dibenzyl tartrate, or tartaric acid) and separating the diasteromers by fractional crystallization followed by neutralization to break the salt, thus providing the corresponding pure enantiomers. All such isomers, including diastereomers, enantiomers and mixtures thereof are considered as part of this invention for all of the compounds of the present invention, including the compounds of the present invention. Also, some of the compounds of this invention are atropisomers (e.g., substituted biaryls) and are considered as part of this invention.

More specifically, the compounds of this invention may be obtained in enantiomerically enriched form by resolving the racemate of the final compound or an intermediate in its synthesis, employing chromatography (preferably high pressure liquid chromatography [HPLC]) on an asymmetric resin (preferably Chiralcel™ AD or OD (obtained from Chiral Technologies, Exton, Pa.)) with a mobile phase consisting of a hydrocarbon (preferably heptane or hexane) containing between 0 and 50% isopropanol (preferably between 2 and 20%) and between 0 and 5% of an alkyl amine (preferably 0.1% of diethylamine). Concentration of the product containing fractions affords the desired materials.

Some of the compounds of this invention are acidic and they form a salt with a pharmaceutically acceptable cation. Some of the compounds of this invention are basic and they form a salt with a pharmaceutically acceptable anion. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds can be obtained in crystalline form by dissolution in an appropriate solvent(s) such as ethanol, hexanes or water/ethanol mixtures.

In addition, when the compounds of this invention form hydrates or solvates they are also within the scope of the invention.

The compounds of this invention, their prodrugs and the salts of such compounds and prodrugs are all adapted to therapeutic use as agents that inhibit cholesterol ester transfer protein activity in mammals, particularly humans. Thus, the compounds of this invention elevate plasma HDL cholesterol, its associated components, and the functions performed by them in mammals, particularly humans. By virtue of their activity, these agents also reduce plasma levels of triglycerides, VLDL cholesterol, Apo-B, LDL cholesterol and their associated components in mammals, particularly humans. Moreover, these compounds are useful in equalizing LDL cholesterol and HDL cholesterol. Hence, these compounds are useful for the treatment and correction of the various dyslipidemias observed to be associated with the development and incidence of atherosclerosis and cardiovascular disease, including coronary artery disease, coronary heart disease, coronary vascular disease, peripheral vascular disease, hypoalphalipoproteinemia, hyperbetalipoproteinemia, hypertriglyceridemia, hypercholesterolemia, familial-hypercholesterolemia, low HDL and associated components, elevated LDL and associated components, elevated Lp(a), elevated small-dense LDL, elevated VLDL and associated components and post-prandial lipemia.

Further, introduction of a functional CETP gene into an animal lacking CETP (mouse) results in reduced HDL levels (Agellon, L. B., et al: J. Biol. Chem. (1991) 266: 10796-10801.) and increased susceptibility to atherosclerosis. (Marotti, K. R., et al: Nature (1993) 364: 73-75.). Also, inhibition of CETP activity with an inhibitory antibody raises HDL-cholesterol in hamster (Evans, G. F., et al: J. of Lipid Research (1994) 35: 1634-1645.) and rabbit (Whitlock, M. E., et al: J. CHn. Invest. (1989) 84: 129-137). Suppression of increased plasma CETP by intravenous injection with antisense oligodeoxynucleotides against CETP mRNA reduced atherosclerosis in cholesterol-fed rabbits (Sugano, M., et al: J. of Biol. Chem. (1998) 273: 5033-5036.) Importantly, human subjects deficient in plasma CETP, due to a genetic mutation possess markedly elevated plasma HDL-cholesterol levels and apolipoprotein A-I, the major apoprotein component of HDL. In addition, most demonstrate markedly decreased plasma LDL cholesterol and apolipoprotein B (the major apolipoprotein component of LDL. (Inazu, A., Brown, M. L., Hesler, C. B., et al.: N. Engl. J. Med. (1990) 323: 1234-1238.)

Given the negative correlation between the levels of HDL cholesterol and HDL associated lipoproteins, and the positive correlation between triglycerides, LDL cholesterol, and their associated apolipoproteins in blood with the development of cardiovascular, cerebral vascular and peripheral vascular diseases, the compounds of this invention, their prodrugs and the salts of such compounds and prodrugs, by virtue of their pharmacologic action, are useful for the prevention, arrestment and/or regression of atherosclerosis and its associated disease states. These include cardiovascular disorders (e.g., angina, ischemia, cardiac ischemia and myocardial infarction), complications due to cardiovascular disease therapies (e.g., reperfusion injury and angioplastic restenosis), hypertension, elevated cardiovascular risk associated with hypertension, stroke, atherosclerosis associated with organ transplantation, cerebrovascular disease, cognitive dysfunction (including, but not limited to, dementia secondary to atherosclerosis, transient cerebral ischemic attacks, neurodegeneration, neuronal deficient, and delayed onset or procession of Alzheimer's disease), elevated levels of oxidative stress, elevated levels of C-Reactive Protein, Metabolic Syndrome and elevated levels of HbAlC.

Because of the beneficial effects widely associated with elevated HDL levels, an agent which inhibits CETP activity in humans, by virtue of its HDL increasing ability, also provides valuable avenues for therapy in a number of other disease areas as well.

Thus, given the ability of the compounds of this invention, their prodrugs and the salts of such compounds and prodrugs to alter lipoprotein composition via inhibition of cholesterol ester transfer, they are of use in the treatment of vascular complications associated with diabetes, lipoprotein abnormalities associated with diabetes and sexual dysfunction associated with diabetes and vascular disease. Hyperlipidemia is present in most subjects with diabetes mellitus (Howard, B. V. 1987. J. Lipid Res. 28, 613). Even in the presence of normal lipid levels, diabetic subjects experience a greater risk of cardiovascular disease (Kannel, W. B. and McGee, D. L. 1979. Diabetes Care 2, 120). CETP-mediated cholesteryl ester transfer is known to be abnormally increased in both insulin-dependent (Bagdade, J. D., Subbaiah, P. V. and Ritter, M. C. 1991. Eur. J. Clin. Invest. 21, 161) and non-insulin dependent diabetes (Bagdade. J. D., Ritter, M. C, Lane, J. and Subbaiah. 1993. Atherosclerosis 104, 69). It has been suggested that the abnormal increase in cholesterol transfer results in changes in lipoprotein composition, particularly for VLDL and LDL, that are more atherogenic (Bagdade, J. D., Wagner, J. D., Rudel, L. L., and Clarkson, T. B. 1995. J. Lipid Res. 36, 759). These changes would not necessarily be observed during routine lipid screening. Thus the present invention will be useful in reducing the risk of vascular complications as a result of the diabetic condition.

The described agents are useful in the treatment of obesity and elevated cardiovascular risk associated with obesity. In both humans (Radeau, T., Lau, P., Robb, M., McDonnell, M., Ailhaud, G. and McPherson, R., 1995. Journal of Lipid Research. 36 (12):2552-61) and nonhuman primates (Quinet, E., Tall, A., Ramakrishnan, R. and Rudel, L., 1991. Journal of Clinical Investigation. 87 (5):1 559-66) mRNA for CETP is expressed at high levels in adipose tissue. The adipose message increases with fat feeding (Martin, L. J., Connelly, P. W., Nancoo, D., Wood, N., Zhang, Z. J., Maguire, G., Quinet, E., Tall, A. R., Marcel, Y. L. and McPherson, R., 1993. Journal of Lipid Research. 34 (3):437-46), and is translated into functional transfer protein and through secretion contributes significantly to plasma CETP levels. In human adipocytes the bulk of cholesterol is provided by plasma LDL and HDL (Fong, B. S., and Angel, A., 1989. Biochimica et Biophysica Acta. 1004 (1):53-60). The uptake of HDL cholesteryl ester is dependent in large part on CETP (Benoist, F., Lau, P., McDonnell, M., Doelle, H., Milne, R. and McPherson, R., 1997. Journal of Biological Chemistry. 272 (38):23572-7). This ability of CETP to stimulate HDL cholesteryl uptake, coupled with the enhanced binding of HDL to adipocytes in obese subjects (Jimenez, J. G., Fong, B., Julien, P., Despres, J. P., Rotstein, L., and Angel, A., 1989. International Journal of Obesity. 13 (5):699-709), suggests a role for CETP, not only in generating the low HDL phenotype for these subjects, but in the development of obesity itself by promoting cholesterol accumulation. Inhibitors of CETP activity that block this process therefore serve as useful adjuvants to dietary therapy in causing weight reduction.

CETP inhibitors are useful in the treatment of inflammation due to Gram-negative sepsis and septic shock. For example, the systemic toxicity of Gram-negative sepsis is in large part due to endotoxin, a lipopoiysaccharide (LPS) released from the outer surface of the bacteria, which causes an extensive inflammatory response. Lipopoiysaccharide can form complexes with lipoproteins (Ulevitch, R. J., Johnston, A. R., and Weinstein, D. B., 1981. J. Clin. Invest. 67, 827-37). In vitro studies have demonstrated that binding of LPS to HDL substantially reduces the production and release of mediators of inflammation (Ulevitch, R J., Johnston, A. R., 1978. J. Clin. Invest. 62, 1313-24). In vivo studies show that transgenic mice expressing human apo-Al and elevated HDL levels are protected from septic shock (Levine, D. M., Parker, T. S., Donnelly, T. M., Walsh, A. M., and Rubin, A. L. 1993. Proc. NatI. Acad. Sci. 90, 12040-44). Importantly, administration of reconstituted HDL to humans challenged with endotoxin resulted in a decreased inflammatory response (Pajkrt, D., Doran, J. E., Koster, F., Lerch, P. G., Amet, B., van der Poll, T., ten Gate, J. W., and van Deventer, S. J. H. 1996. J. Exp. Med. 184, 1601-08). The CETP inhibitors, by virtue of the fact that they raise HDL levels, attenuate the development of inflammation and septic shock. These compounds would also be useful in the treatment of endotoxemia, autoimmune diseases and other systemic disease indications, organ or tissue transplant rejection and cancer.

The utility of the compounds of the invention, their prodrugs and the salts of such compounds and prodrugs as medical agents in the treatment of the above described disease/conditions in mammals (e.g. humans, male or female) is demonstrated by the activity of the compounds of this invention in conventional assays and the in vivo assay described below. The in vivo assay (with appropriate modifications within the skill in the art) may be used to determine the activity of other lipid or triglyceride controlling agents as well as the compounds of this invention. Such assays also provide a means whereby the activities of the compounds of this invention, their prodrugs and the salts of such compounds and prodrugs (or the other agents described herein) can be compared to each other and with the activities of other known compounds. The results of these comparisons are useful for determining dosage levels in mammals, including humans, for the treatment of such diseases.

The following protocols can of course be varied by those skilled in the art.

The hyperalphacholesterolemic activity of the compounds can be determined by assessing the effect of these compounds on the action of cholesteryl ester transfer protein by measuring the relative transfer ratio of radiolabeled lipids between lipoprotein fractions, essentially as previously described by Morton in J. Biol. Chem. 256, 11992, 1981 and by Dias in Clin. Chem. 34, 2322, 1988.

### CETP In Vitro Assay

The following is a brief description of assays of cholesteryl ester transfer in 97% (whole) or diluted human plasma (Zn vitro) and animal plasma (ex vivo): CETP activity in the presence or absence of drug is assayed by determining the transfer of ³H-labeled cholesteryl oleate (CO) from exogenous tracer HDL or LDL to the nonHDL or HDL lipoprotein fraction in human plasma, respectively, or from ³H-labeled LDL to the HDL fraction in animal plasma. Labeled human lipoprotein substrates are prepared similarly to the method described by Morton in which the endogenous CETP activity in plasma is employed to transfer ³H—CO from phospholipid liposomes to all the lipoprotein fractions in plasma. ³H-labeled LDL and HDL are subsequently isolated by sequential ultracentrifugation at the density cuts of 1.019-1.063 and 1.10-1.21 g/ml, respectively.

For the 97% or whole plasma activity assay, ³H-labeled HDL is added to plasma at 10-25 nmoles CO/ml and the samples incubated at 37° C. for 2.5-3 hrs. Non-HDL lipoproteins are then precipitated by the addition of an equal volume of 20% (wt/vol) polyethylene glycol 8000 (Dias). The samples are centrifuged 750 g×20 minutes and the radioactivity contained in the HDL-containing supernatant determined by liquid scintillation counting. Introducing varying quantities of the compounds of this invention as a solution in dimethylsulfoxide into human plasma, before addition of the radiolabeled cholesteryl oleate, and comparing the amounts of radiolabel transferred compared to incubations containing no inhibitor compounds allows the cholesteryl ester transfer inhibitory activities to be determined.

When a more sensitive assay is desirable, an in vitro assay using diluted human plasma is utilized. For this assay, ³H-labeled LDL is added to plasma at 50 nmoles CO/ml and the samples incubated at 37° C. for 7 hrs. Non-HDL lipoproteins are then precipitated by the addition of potassium phosphate to 100 mM final concentration followed by manganese chloride to 20 m M final concentration. After vortexing, the samples are centrifuged 750 g×20 minutes and the radioactivity contained in the HDL-containing supematant determined by liquid scintillation counting. Introducing varying quantities of the compounds of this invention as a solution in dimethylsulfoxide into diluted human plasma, before addition of the radiolabeled cholesteryl oleate, and comparing the amounts of radiolabel transferred compared to incubations containing no inhibitor compounds allows the cholesteryl ester transfer inhibitory activities to be determined. This assay has been adapted to run in microtiter plate format with liquid scintillation counting accomplished using a Wallac plate reader.

### CETP In Vivo Assay

Activity of these compounds in vivo can be determined by the amount of agent required to be administered, relative to control, to inhibit cholesteryl ester transfer activity by 50% at various time points ex vivo or to elevate HDL cholesterol by a given percentage in a CETP-containing animal species. Transgenic mice expressing both human CETP and human apolipoprotein Al (Charles River, Boston, Mass.) may be used to assess compounds in vivo. The compounds to be examined are administered by oral gavage in an emulsion vehicle containing 20% (v:v) olive oil and 80% sodium taurocholate (0.5%). Blood is taken from mice retroorbitally before dosing, if a predose blood sample is desirable. At various times after dosing, ranging from 4 h to 24 h, the animals are sacrificed, blood obtained by heart puncture, and lipid parameters measured, including total cholesterol, HDL and LDL cholesterol, and triglycerides. CETP activity is determined by a method similar to that described above except that ³H-cholesteryl oleate-containing LDL is used as the donor source as opposed to HDL. The values obtained for lipids and transfer activity are compared to those obtained prior to dosing and/or to those from mice receiving vehicle alone.

### Plasma Lipids Assay

The activity of these compounds may also be demonstrated by determining the amount of agent required to alter plasma lipid levels, for example HDL cholesterol levels, LDL cholesterol levels, VLDL cholesterol levels or triglycerides, in the plasma of certain mammals, for example marmosets that possess CETP activity and a plasma lipoprotein profile similar to that of humans (Crook et al. Arteriosclerosis 10, 625, 1990). Adult marmosets are assigned to treatment groups so that each group has a similar mean±SD for total, HDL, and/or LDL plasma cholesterol concentrations. After group assignment, marmosets are dosed daily with compound as a dietary admix or by intragastric intubation for from one to eight days. Control marmosets receive only the dosing vehicle. Plasma total, LDL VLDL and HDL cholesterol values can be determined at any point during the study by obtaining blood from an antecubital vein and separating plasma lipoproteins into their individual subclasses by density gradient centrifugation, and by measuring cholesterol concentration as previously described (Crook et al. Arteriosclerosis 10, 625, 1990).

### In Vivo Atherosclerosis Assay

Anti-atherosclerotic effects of the compounds can be determined by the amount of compound required to reduce the lipid deposition in rabbit aorta. Male New Zealand White rabbits are fed a diet containing 0.2% cholesterol and 10% coconut oil for 4 days (meal-fed once per day). Rabbits are bled from the marginal ear vein and total plasma cholesterol values are determined from these samples. The rabbits are then assigned to treatment groups so that each group has a similar mean±SD for total plasma cholesterol concentration, HDL cholesterol concentration, triglyceride concentration and/or cholesteryl ester transfer protein activity. After group assignment, rabbits are dosed daily with compound given as a dietary admix or on a small piece of gelatin based confection. Control rabbits receive only the dosing vehicle, be it the food or the gelatin confection. The cholesterol/coconut oil diet is continued along with the compound administration throughout the study. Plasma cholesterol values and cholesteryl ester transfer protein activity can be determined at any point during the study by obtaining blood from the marginal ear vein. After 3-5 months, the rabbits are sacrificed and the aortae are removed from the thoracic arch to the branch of the iliac arteries. The aortae are cleaned of adventitia, opened longitudinally and then analyzed unstained or stained with Sudan IV as described by Holman et. al. (Lab. Invest. 1958, 7,42-47). The percent of the lesioned surface area is quantitated by densitometry using an Optimas Image Analyzing System (Image Processing Systems). Reduced lipid deposition is indicated by a reduction in the percent of lesioned surface area in the compound-receiving group in comparison with the control rabbits.

### Antiobesity Protocol

The ability of CETP inhibitors to cause weight loss can be assessed in obese human subjects with body mass index (BMI)≧30 kg/m². Doses of inhibitor are administered sufficient to result in an increase of ≧25% in HDL cholesterol levels. BMI and body fat distribution, defined as waist (W) to hip (H) ratio (WHR), are monitored during the course of the 3-6 month studies, and the results for treatment groups compared to those receiving placebo.

### In Vivo Sepsis Assay4

In vivo studies show that transgenic mice expressing human apo-AI and elevated HDL levels are protected from septic shock. Thus the ability of CETP inhibitors to protect from septic shock can be demonstrated in transgenic mice expressing both human apo-Al and human CETP transgenes (Levine, D. M., Parker, T. S., Donnelly, T. M., Walsh, A. M. and Rubin, A. L., 1993. Proc. Natl. Acad. Sci. 90, 12040-44). LPS derived from E. coli is administered at 30 mg/kg by Lp. injection to animals which have been administered a CETP inhibitor at an appropriate dose to result in elevation of HDL. The number of surviving mice is determined at times up to 48 h after LPS injection and compared to those mice administered vehicle (minus CETP inhibitor) only.

Administration of the compounds of this invention can be via any method which delivers a compound of this invention systemically and/or locally. These methods include oral routes, parenteral, intraduodenal routes, etc. Generally, the compounds of this invention are administered orally, but parenteral administration (e.g., intravenous, intramuscular, subcutaneous or intramedullary) may be utilized, for example, where oral administration is inappropriate for the target or where the patient is unable to ingest the drug.

In general an amount of a compound of this invention is used that is sufficient to achieve the therapeutic effect desired (e.g., HDL elevation).

In general an effective dosage for the compounds of this invention is about 0.001 to 100 mg/kg/day of the compound, a prodrug thereof, or a pharmaceutically acceptable salt of said compound or of said prodrug. An especially preferred dosage is about 0.01 to 10 mg/kg/day of the compound, a prodrug thereof, or a pharmaceutically acceptable salt of said compound or of said prodrug.

A dosage of the combination pharmaceutical agents to be used in conjuction with the CETP inhibitors is used that is effective for the indication being treated.

For example, typically an effective dosage for HMG-CoA reductase inhibitors is in the range of 0.01 to 100 mg/kg/day. In general an effect dosage for a PPAR modulator is in the range of 0.01 to 100 mg/kg/day.

The compounds of the present invention are generally administered in the form of a pharmaceutical composition comprising at least one of the compounds of this invention together with a pharmaceutically acceptable vehicle, diluent or carrier as described below. Thus, the compounds of this invention can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. A preferred formulation is a solution or suspension in an oil, for example, a vegetable oil, such as olive oil; triglycerides such as those marketed under the name, Miglyol™; or mono- or diglycerides such as those marketed under the name, Capmul™, for example, in a soft gelatin capsule. Antioxidants may be added to prevent long-term degradation as appropriate. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

Pharmaceutical compositions comprising a solid amorphous dispersion of a cholesteryl ester transfer protein (CETP) inhibitor and a concentration-enhancing polymer are described in International Publication No. WO 02/11710, which is hereby incorporated by reference herein. Self-emulsifying formulations of cholesteryl ester transfer protein (CETP) inhibitors are described in International Publication No. WO 03/000295, which is hereby incorporated by reference herein. Methods for depositing small drug crystals on excipients are set forth in the literature, such as in J. Pharm. Pharmacol. 1987, 39:769-773, which is hereby incorporated by reference herein.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g. topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition orformulation to be administered will contain a quantity of a compound(s) according to the invention in an amount effective to treat the disease/condition of the subject being treated, e.g., atherosclerosis.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of the present invention, a prodrug thereof or a salt of such compound or prodrug and a second compound as described above. The kit comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc . . . . Second Week, Monday, Tuesday, . . . ” etc. Other variations of memory aids will be readily apparent. A “daily dose” can be a single tablet or capsule or several pills or capsules to be taken on a given day. Also, a daily dose of compounds of the present invention can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of this invention either alone or in combination with each other or other compounds generally will be administered in a convenient formulation. The following formulation examples only are illustrative and are not intended to limit the scope of the present invention.

In the formulations which follow, “active ingredient” means a compound of this invention.

Formulation 1: Gelatin Capsules

Hard gelatin capsules are prepared using the following:

| Ingredient | Quantity (mg/capsule) |
|---|---|
| Active ingredient | 0.25-100 |
| Starch, NF | 0-650 |
| Starch flowable powder | 0-50 |
| Silicone fluid 350 centistokes | 0≧15 |

A tablet formulation is prepared using the ingredients below:

Formulation 2: Tablets

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-100 mg of active ingredients are made up as follows:

Formulation 3: Tablets

| Ingredient | Quantity (mg/tablet) |
|---|---|
| Active ingredient | 0.25-100 |
| Starch | 45 |
| Cellulose, microcrystalline | 35 |
| Polyvinylpyrrolidone (as 10% solution in water) | 4 |
| Sodium carboxymethyl cellulose | 4.5 |
| Magnesium stearate | 0.5 |
| Talc | 1 |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 ml dose are made as follows:

Formulation 4: Suspensions

| Ingredient | Quantity (mg/5 ml) | |
|---|---|---|
| Active ingredient | 0.25-100 | mg |
| Sodium carboxymethyl cellulose | 50 | mg |
| Syrup | 1.25 | mg |
| Benzoic acid solution | 0.10 | mL |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form smooth paste. The benzoic acid solution, flavor, and color are diluted with some of the water and added, with stirring. Sufficient water is then added to produce the required volume.

An aerosol solution is prepared containing the following ingredients:

Formulation 5: Aerosol

| Ingredient | Quantity (% by weight) |
|---|---|
| Active ingredient | 0.25 |
| Ethanol | 25.75 |
| Propellant 22 (Chlorodifluoromethane) | 70.00 |

The active ingredient is mixed with ethanol and the mixture added to a portion of the propellant 22, cooled to 30° C., and transferred to a filling device. The required amount is then fed to a stainless steel container and diluted with the remaining propellant. The valve units are then fitted to the container.

Suppositories are prepared as follows:

Formulation 6: Suppositories

| Ingredient | Quantity (mg/suppository) |
|---|---|
| Active ingredient | 250 |
| Saturated fatty acid glycerides | 2,000 |

The active ingredient is passed through a No. 60 mesh U.S. sieve and suspended in the saturated fatty acid glycerides previously melted using the minimal necessary heat. The mixture is then poured into a suppository mold of nominal 2 g capacity and allowed to cool.

An intravenous formulation is prepared as follows:

Formulation 7: Intravenous Solution

| Ingredient | Quantity | |
|---|---|---|
| Active ingredient dissolved in ethanol 1% | 20 | mg |
| Intralipid ™ emulsion | 1,000 | mL |

The solution of the above ingredients is intravenously administered to a patient at a rate of about 1 ml_per minute.

Soft gelatin capsules are prepared using the following:

Formulation 8: Soft Gelatin Capsule With Oil Formulation

| Ingredient | Quantity (mg/capsule) |
|---|---|
| Active ingredient | 10-500 |
| Olive Oil or Miglyol ™ Oil | 500-1000 |

The active ingredient above may also be a combination of agents.

### General Experimental Procedures

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in 0° C. or is at ambient temperature, and pressure is at or near atmospheric. Commercial reagents were utilized without further purification. Room or ambient temperature refers to 20-25° C. All non-aqueous reactions were run under a nitrogen atmosphere for convenience and to maximize yields. Concentration in vacuo means that a rotary evaporator was used. The names for the compounds of the invention were created by the Autonom 2.0 PC-batch version from Beilstein Informationssysteme GmbH (ISBN 3-89536-976-4). The chemical structures depicted may be only exemplary of the general structure or of limited isomers, and not include specific stereochemistry as recited in the chemical name.

NMR spectra were recorded on a Varian Unity 400 (Varian Co., Palo Alto, Calif.) NMR spectrometer at ambient temperature. Chemical shifts are expressed in parts per million (δ) relative to an external standard (tetramethylsilane). The peak shapes are denoted as follows: s, singlet; d, doublet, t, triplet, q, quartet, m, multiplet with the prefix br indicating a broadened signal. The coupling constant (J) data given have a maximum error of ±0.41 Hz due to the digitization of the spectra that are acquired. Mass spectra were obtained by (1) atmospheric pressure chemical ionization (APCI) in alternating positive and negative ion mode using a Fisons Platform II Spectrometer or a Micromass MZD Spectrometer (Micromass, Manchester, UK) or (2) electrospray ionization in alternating positive and negative ion mode using a Micromass MZD Spectrometer (Micromass, Manchester, UK) with a Gilson LC-MS interface (Gilson Instruments, Middleton, Wis.) or (3) a QP-8000 mass spectrometer (Shimadzu Corporation, Kyoto, Japan) operating in positive or negative single ion monitoring mode, utilizing electrospray ionization or atmospheric pressure chemical ionization. Where the intensity of chlorine- or bromine-containing ions are described, the expected intensity ratio was observed (approximately 3:1 for ³⁵Cl/³⁷Cl-containing ions and 1:1 for ⁷⁹Br/⁸¹Br-containing ions) and the position of only the lower mass ion is given.

Column chromatography was performed with either Baker Silica Gel (40 μm) (J. T. Baker, Phillipsburg, N.J.) or Silica Gel 60 (40-63 μm) (EM Sciences, Gibbstown, N.J.). Flash chromatography was performed using a Flash 12 or Flash 40 column (Biotage, Dyar Corp., Charlottesville, Va.). Radial chromatography was performed using a chromatotron Model 7924T (Harrison Research, Palo Alto, Calif.). Preparative HPLC purification was performed on a Shimadzu 10A preparative HPLC system (Shimadzu Corporation, Kyoto, Japan) using a model SIL-10A autosampler and model 8A HPLC pumps. Preparative HPLC-MS was performed on an identical system, modified with a QP-8000 mass spectrometer operating in positive or negative single ion monitoring mode, utilizing electrospray ionization or atmospheric pressure chemical ionization. Elution was carried out using water/acetonitrile gradients containing either 0.1% formic acid or ammonium hydroxide as a modifier. In acidic mode, typical columns used include Waters Symmetry C8, 5 μm, 19×50 mm or 30×50 mm, Waters XTerra C18, 5 μm, 50×50 (Waters Corp, Milford, Mass.) or Phenomenex Synergi Max-RP 4 μm, 50×50 mm (Phenomenex Inc., Torrance, Calif.). In basic mode, the Phenomenex Synergi Max-RP 4 μm, 21.2×50 mm or 30×50 mm columns (Phenomenex Inc., Torrance, Calif.) were used.

Optical rotations were determined using a Jasco P-1020 Polarimeter Jasco Inc., Easton, Md.)

Dimethylformamide (“DMF”), tetrahydrofuran (“THF”), toluene and dichloromethane (“DCM”) were the anhydrous grade supplied by Aldrich Chemical Company (Milwaukee, Wis.). Unless otherwise specified, reagents were used as obtained from commercial sources. The terms “concentrated” and “evaporated” refer to removal of solvent at 1-200 mm of mercury pressure on a rotary evaporator with a bath temperature of less than 45° C. The abbreviation “min” stand for “minutes” and “h” or “hr” stand for “hours.” The abbreviation “gm” or “g” stand for grams. The abbreviation “μl” or “μL” stand for microliters.

### Preparation 1

### (2R.4S) and (2R.4f?)-2-Ethyl-4-hvdroxy-6-trifluoromethyl-3,4-dihydro-2H-quinoline-1-carboxylic-acid/so-propyl ester

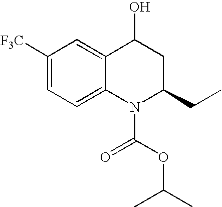

To a solution of (R)-[4-(2-ethyl-4-oxo-6-trifluoromethyl-3,4-dihydro-2H-quinoline-1-carboxylic-acid/sopropyl ester (1.38 g, 4.22 mmol) in ethanol (17 ml_) at 0° C. was added solid sodium borohydride (239 mg, 6.33 mmol). After 10 minutes, acetone was added to quench the reaction and the mixture was concentrated under vacuum. The residue was dissolved in ethyl acetate and the solution was washed with a brine solution. The organic layer was dried over anhydrous sodium sulfate, diluted with toluene and evaporated to dryness to give the title compound as a 6:1 mixture of diastereoisomers which were carried forward unseparated. MS: 332 [M+Hf

¹H-NMR (CDCl₃) δ: 7.79 (s, 1H), 7.60 (d 1H), 6.45 (d, 1H), 5.10 (m, 1H), 4.65 (m, 1H), 4.44 (m, 1H), 4.25 (m, 1H), 3.40 (brs, 1H), 2.65 (m, 1H), 1.75 (m, 1H)₁ 1.44 (d, 3H), 1.40 (d, 3H), 0.96 (t, 3H).

### Preparation 2

### (2R. 4S) and (2R, 4f?)-2-Ethyl-4-chloro-6-trifluoromethyl-3,4-dihydro-2H-quinoline-1-carboxylic-acid/so-propyl ester

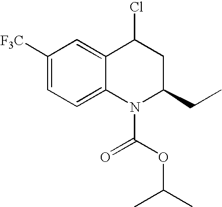

(2R,4S) and (2R,4R)-2-Ethyl-4-hydroxy-6-trifluoromethyl-3,4-dihydro-2H-quinoline-1-carboxylic-acid/so-propyl ester from preparation 1 (1.3 g, 4.16 mmol) was cooled to 0° C. and SOCl₂ (6.6 ml) was added. The mixture was stirred at ambient temperature for 1 hour. The reaction was then quenched by the addition of water. The solution was saturated with aqueous NaHCO₃and extracted three times with ethyl acetate. The organic layers were collected, dried over anhydrous sodium sulfate and evaporated to dryness to give title compound (1.43 g) which typically was carried on without further purification. MS: 350 [M+H]⁺ found

¹H-NMR (CDCl₃) δ: 7.82 (s, 1H), 7.67 (d 1H), 6.45 (d, 1H), 5.10 (m, 2H), 4.65 (m, 1H), 4.44 (m, 1H), 4.25 (m, 1H), 2.75 (m, 1H), 2.1 (m, 1H), 1.35 (d, 3H), 1.30 (d, 3H), 0.96 (t, 3H). Preparation 3: Benzhvdrylidene-(3,5-bis-trifluoromethyl-benzyl)-amine

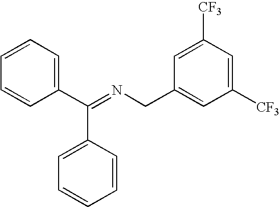

A solution of 3,5-bis-trifluoromethyl-benzylamine (75 gm, 0.308 mol) and benzophenone imine (53.5 mL, 57.78 gm, 0.319 mol) in diisopropyl ether (375 mL) was heated under reflux for lhr. The mixture was then concentrated under vacuum to a volume of 100 mL, isopropanol (250 mL) was added and the volume reduced to 150 mL at atmospheric pressure. After stirring overnight the crystalline product was isolated by filtration and washed with isopropanol (25 mL) to give the title compound (92%).

### Preparation 4

### [(2R,4R,4aS) and (2R4R4af?)I-4-r(Benzhvdrylidene-aminoV(3,5-bis-trifluoromethyl-phenyl)-methyll-2-ethyl-6-trifluoromethyl-3.4-dihvdro-2H-quinoline-1-carboxylic acid isopropyl ester

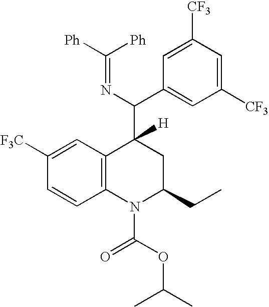

To a solution of (2R.4S) and (2R4R;)-4-chloro-2-ethyl-6-trifluoromethyl-3,4-dihydro-2H-quinoline-1-carboxylic acid iso-propyl ester (1.42 gm, 0.4.06 mmol) and benzhydrylidene-(3,5-bis-trifluoromethyl-benzyl)-amine (1.65 gm, 0.4.06 mmol) in N,N-dimethylformamide (13.3 mL) was added a solution of sodium hexamethyidisilazide (1M in tetrahydrofuran, 5.28 mL, 5.28 m mol) under nitrogen. The mixture was stirred at 20° C. to 30° C. for 20 minutes then water followed by ethyl acetate was added. The organic layer was collected, dried over magnesium sulfate and concentrated to dryness. The crude mixture of isomers was purified by chromatography with silica using 96:4 hexanes: ethyl acetate to afford the title compound in 35% yield (0.50 gm). MS: 721 [M+H]⁺

¹H-NMR (CDCl₃) δ: 7.70 (s, 1H), 7.40 (br s, 11H), 6.85 (d, 1H), 6.40 (s, 1H), 4.9 (m, 1H), 4.66 (d, 1H), 4.25 (m, 1H), 3.40 (br s, 1H), 2.75 (m, 1H), 1.65 (m, 1H), 1.54 (m, 1H), 1.40 (m, 1H), 1.39 (d, 3H), 1.05 (d, 3H), 0.96 (t, 3H).

### Example 1

### \(2R.AR,4aS)1-4-rAmino-(3.5-bis-trifluoromethyl-phenvD-methyl1-2-ethyl-6-trifluoromethyl-3.4-dihvdro-2H-quinoline-1-carboxylic acid isopropyl ester

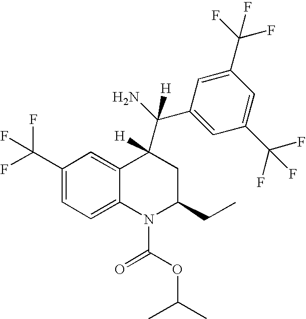

To a suspension of [(2R,4f?,4aS)]-4-[(benzhydrylidene-amino)-(3,5-bis-trifluoromethyl-phenyl)-methyllˆ-ethyl-6-trifluoromethyl-Sˆ-dihydroˆH-quinoline-i-carboxylic acid isopropylester (0.70 mmol) in THF (10 mL) was added 5 mL of 2N HCl solution. The reaction was stirred at 20 to 30° C. for 1 hour. The reaction was concentrated to dryness, quenched with saturated aqueous NaHCO₃ and extracted 3 times with ethyl acetate. The organic layers were collected, dried over sodium sulfate, filtered and concentrated to dryness to provide a crude mixture which was purified by chromatography on silica to provide the title compound in 88% yield (0.34 g). MS: 556 [M+Hf

¹H-NMR (CDCl₃) δ: 7.70 (s, 1H), 7.61 (s, 2H), 7.55 (d, 1H), 7.40 (d, 1H), 6.59 (s, 1H), 5.15 (m, 1H), 4.68 (m, 1H), 4.10 (d, 1H), 2.95 (m, 1H), 1.75 (m, 1H), 1.60 (m, 1H), 1.40 (d, 3H), 1.35 (d, 3H), 0.95 (t, 3H). Examples 2-10 were prepared using a procedure analogous to example 1 with the appropriate starting materials.

### Example 2

### (2f?. AR,?4aS)-4-rAmino-(3,5-bis(trifluoromethyl-phenyl)-methvn-2-ethyl-6-trifluoromethyl-3,4-dihvdro-2H-quinoline-1-carboxylic acid n-propyl ester

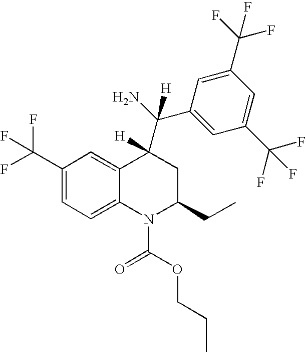

MS: 557 [M+H]⁺

¹H-NMR (CDCl₃) δ: 7.78 (s, 1H), 7.65 (s, 1H), 7.42 (d, 1H), 7.01 (s, 1H), 4.52 (m, 2H), 4.20 (m, 1H), 3.00 (br m, 1H), 2.51 (br m, 1H), 1.77 (m, 2H), 1.45 (m, 2H), 0.99 (t, 3H), 0.86 (t, 3H)

### Example 3

### (2R,AR,4aS)-4-rAmino-(3,5-bis(trifluoromethyl-phenyl)-methvn-2-ethyl-6-trifluoromethyl-3,4-dihydro-2H-quinoline-1-carboxylic acid isobutyl ester

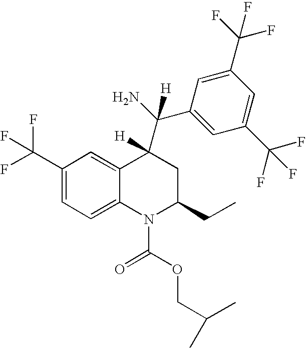

MS: 571 [M+Hf

¹H-NMR (CDCl₃) δ: 7.78 (s, 1H), 7.61 (s, 1H), 7.57 (d, 1H), 7.40 (s, 1H), 4.60 (m, 2H), 4.18 (m, 1H), 4.08 (d, 2H), 2.98 (m, 1H), 2.81 (m, 1H), 2.00 (m, 1H), 1.69 (m, 2H), 1.57 (m, 2H), 1.00 (t, 3H), 0.87 (t, 3H)

### Example 4

### (2S,4R, 4aS)-4-rAmino-(3,5-bis(trifluoromethyl-phenyl)-methv π-2-cydopropyl-6-trifluoromethyl-3,4-dihvdro-2/-/-quinoline-1-carboxylic acid isopropyl ester

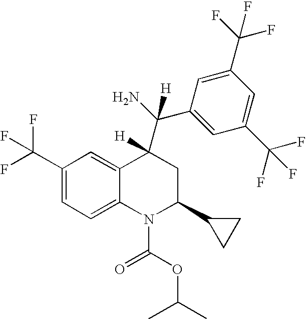

MS: 569 [M+H]⁺

¹H-NMR (CDCl₃) δ: 7.70 (s, 1H), 7.61 (s, 2H), 7.57 (d, 1H), 7.40 (d, 1H), 6.62 (s, 1H), 5.15 (m, 1H), 4.28 (m, 1H), 4.15 (m, 1H), 3.03 (m, 1H), 2.95 (m, 1H), 1.89 (m, 1H), 1.39 (d, 3H), 1.35 (d, 3H), 0.95 (m, 1H), 0.4-0.2 (m, 4H)

### Example 5

### (2R.AR,4aS)-4-fAmino-3,5-bis(trifluoromethyl-phenyl)-methvn-2-methyl-6-trifluoromethyl-3,4-dihvdro-2/-/-αuinoline-1-carboxylic acid ethyl ester

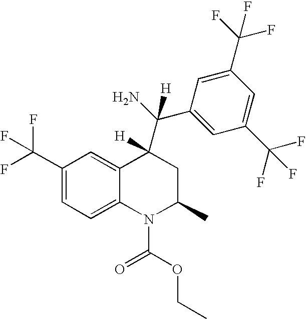

MS: 529 [M+H]⁺

¹H-NMR (CDCl₃) δ: 7.75 (s, 1H), 7.62 (s, 2H), 7.60 (s, 1H), 7.40 (d, 1H), 6.95 (s, 1H), 4.74 (m, 1H), 4.44 (m, 1H), 4.25 (q, 2H), 2.95 (m, 1H), 2.60 (m, 1H), 1.65 (m, 2H), 1.39 (t, 3H), 1.22 (d, 3H)

### Example 6

### (2R,4R,4aS)-4-rAmino-(3,5-bis(trifluoromethyl-phenyly)-methvn-2-methyl-6-trifluoromethyl-3,4-dihvdro-2H-quinoline-1-carboxylic acid isopropyl ester

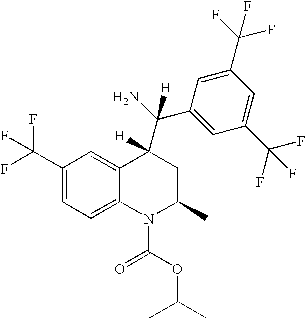

MS: 543 [M+H

¹H-NMR (CDCl₃) δ: 7.75 (s, 1H), 7.62 (s, 2H), 7.60 (s, 1H), 7.40 (d, 1H), 6.95 (s, 1H), 5.15 (m, 1H), 4.74 (m, 1H), 4.40 (d, 1H), 2.95 (m, 1H), 2.64 (m, 1H), 1.65 (m, 2H), 1.40 (d, 3H), 1.30 (d, 3H), 1.22 (d, 3H)

### Example 7

### (2f?,AR.4aS)-4-fAmino-(3,5-bis(trifluoromethyl-phenvl)-methvn-2-methyl-6-trifluoromethyl-3,4-dihydro-2/-/-αuinoline-1-carboxylic acid n-propyl ester

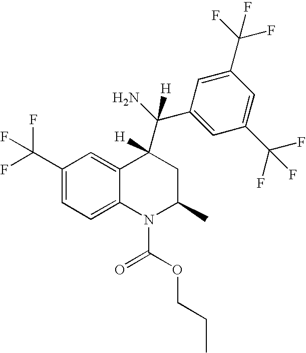

MS: 543 [M+H]⁺

¹H-NMR (CDCl₃) δ: 7.75 (s, 1H), 7.62 (s, 2H), 7.60 (s, 1H), 7.40 (d, 1H), 6.96 (s, 1H), 4.66 (m, 1H), 4.45 (d, 1H), 4.10 (t, 2H), 2.95 (m, 1H), 2.65 (m, 1H), 1.75 (m, 2H), 1.40 (m, 2H), 1.22 (d, 3H), 0.95 (t, 3H)

### Example 8

### (2R,4R,4aS)-4-rAmino-(3,5-bis(trifluoromethyl-phenylVmethvn-2-methyl-6-trifluoronnethyl-3.4-dihvdro-2f/-quinoline-1-carboxylic acid f-butyl ester

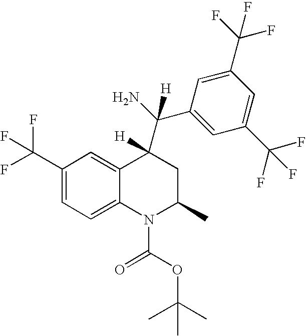

MS: 557 [M+Hf

¹H-NMR (CDCl₃) δ: 7.78 (s, 1H)₁7.59 (s, 2H), 7.50 (s, 1H), 7.39 (d, 1H), 6.76 (s, 1H), 4.66 (m, 1H), 4.45 (d, 1H), 2.95 (m, 1H), 2.65 (m, 1H), 1.60 (s, 9H), 1.22 (d, 3H).

### Example 9

### (2R,4R.4aS)-4-rAmino-3.5-bis(chloro-phenyl)-methvn-2-ethyl-6-trifluoromethyl-3,4-dihvdro-2H-αuinoline-1-carboxylic acid ethyl ester

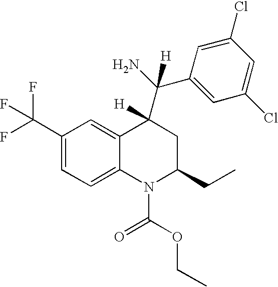

MS: 475.2 [M+H]⁺ found (³⁵Cl₂)

¹H-NMR (CDCl₃) δ: 7.63 (d, J=8.30 Hz, 1H), 7.42 (dd, J=8.30, 1.66Hz, 1H), 7.21 (m, 1H), 7.16 (m, 2H), 4.51 (m, 1H), 4.38 (d, J=5.80 Hz, 1H), 4.29-4.23 (m, 2H), 3.02 (m, 1H), 2.42 (m, 1H), 1.47-1.38 (m, 2H), 1.32 (m, 1H), 1.32 (t, J=7.46 Hz, 3H), 0.80 (t, 7.47 Hz, 3H)

### Example 10

### (2R.4R.4aS)-4-rAmino-3-chloro-5-trifluoromethyl-phenyl)-methyll-2-ethyl-6-trifluoromethyl-3,4-dihvdro-2H-quinoline-1-carboxylic acid ethyl ester

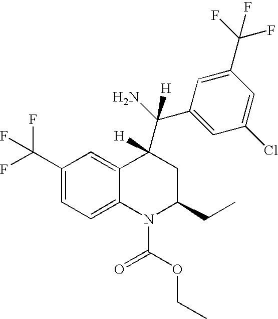

MS: 509.2 [M+H]⁺ found (³⁵Cl)

1 H-NMR (CDCl₃) δ: 7.61 (d, J=8.30 Hz, 1H), 7.48 (s, 1H) 1 7.45 (s, 1H), 7.42 (dd, J=8.30, 1.66 Hz, 1H), 7.31 (s, 1H)₁7.14 (s, 1H), 4.52 (m, 1H), 4.44 (d, J=6.64 Hz, 1H), 4.30-4.23 (m, 2H), 3.01 (m, 1H), 2.48 (m, 1H), 1.50-1.40 (m, 2H)₁ 1.38 (m, 1H)₁ 1.31 (t, J=7.47 Hz, 3H), 0.80 (t, 7.47 Hz ₁ 3H)

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound selected from the group consisting of:
4-[Amino-(3,5-bis-trifluoromethyl-phenyl)-methyl]-2-ethyl-6-trifluoromethyl-3,4-dihydro-2H-quinoline-1-carboxylic acid isopropyl ester;
(2R,4R,4aS)-4-[Amino-(3,5-bis-trifluoromethyl-phenyl)-methyl]-2-ethyl-6-trifluoromethyl-3,4-dihydro-2H-quinoline-1-carboxylic acid isopropyl ester;
(2R,4S,4aS)-4-[Amino-(3,5-bis-trifluoromethyl-phenyl)-methyl]-2-ethyl-6-trifluoromethyl-3,4-dihydro-2H-quinoline-1-carboxylic acid isopropyl ester;
4-[Amino-(3,5-bis(trifluoromethyl-phenyl)-methyl]-2-ethyl-6-trifluoromethyl-3,4-dihydro-2H-quinoline-1-carboxylic acid n-propyl ester;
(2R,4R,4aS)-4-[Amino-(3,5-bis(trifluoromethyl-phenyl)-methyl]-2-ethyl-6-trifluoromethyl-3,4-dihydro-2H-quinoline-1-carboxylic acid n-propyl ester;
(2R,4S,4aS)-4-[Amino-(3,5-bis(trifluoromethyl-phenyl)-methyl]-2-ethyl-6-trifluoromethyl-3,4-dihydro-2H-quinoline-1-carboxylic acid n-propyl ester;
4-[Amino-(3,5-bis(trifluoromethyl-phenyl)-methyl]-2-ethyl-6-trifluoromethyl-3,4-dihydro-2H-quinoline-1-carboxylic acid isobutyl ester;
(2R,4R,4aS)-4-[Amino-(3,5-bis(trifluoromethyl-phenyl)-methyl]-2-ethyl-6-trifluoromethyl-3,4-dihydro-2H-quinoline-1-carboxylic acid isobutyl ester;
(2R,4S,4aS)-4-[Amino-(3,5-bis(trifluoromethyl-phenyl)-methyl]-2-ethyl-6-trifluoromethyl-3,4-dihydro-2H-quinoline-1-carboxylic acid isobutyl ester;
4-[Amino-(3,5-bis(trifluoromethyl-phenyl)-methyl]-2-cyclopropyl-6-trifluoromethyl-3,4-dihydro-2H-quinoline-1-carboxylic acid isopropyl ester;
(2S,4R,4aS)-4-[Amino-(3,5-bis(trifluoromethyl-phenyl)-methyl]-2-cyclopropyl-6-trifluoromethyl-3,4-dihydro-2H-quinoline-1-carboxylic acid isopropyl ester;
(2S,4S,4aS)-4-[Amino-(3,5-bis(trifluoromethyl-phenyl)-methyl]-2-cyclopropyl-6-trifluoromethyl-3,4-dihydro-2H-quinoline-1-carboxylic acid isopropyl ester;
4-[Amino-(3,5-bis(trifluoromethyl-phenyl)-methyl]-2-methyl-6-trifluoromethyl-3,4-dihydro-2H-quinoline-1-carboxylic acid ethyl ester;
(2R,4R,4aS)-4-[Amino-(3,5-bis(trifluoromethyl-phenyl)-methyl]-2-methyl-6-trifluoromethyl-3,4-dihydro-2H-quinoline-1-carboxylic acid ethyl ester;
(2R,4S,4aS)-4-[Amino-(3,5-bis(trifluoromethyl-phenyl)-methyl]-2-methyl-6-trifluoromethyl-3,4-dihydro-2H-quinoline-1-carboxylic acid ethyl ester;
4-[Amino-(3,5-bis(trifluoromethyl-phenyl)-methyl]-2-methyl-6-trifluoromethyl-3,4-dihydro-2H-quinoline-1-carboxylic acid isopropyl ester;
(2R,4R,4aS)-4-[Amino-(3,5-bis(trifluoromethyl-phenyl)-methyl]-2-methyl-6-trifluoromethyl-3,4-dihydro-2H-quinoline-1-carboxylic acid isopropyl ester;
(2R,4S,4aS)-4-[Amino-(3,5-bis(trifluoromethyl-phenyl)-methyl]-2-methyl-6-trifluoromethyl-3,4-dihydro-2H-quinoline-1-carboxylic acid isopropyl ester;
4-[Amino-(3,5-bis(trifluoromethyl-phenyl)-methyl]-2-methyl-6-trifluoromethyl-3,4-dihydro-2H-quinoline-1-carboxylic acid n-propyl ester;
(2R,4R,4aS)-4-[Amino-(3,5-bis(trifluoromethyl-phenyl)-methyl]-2-methyl-6-trifluoromethyl-3,4-dihydro-2H-quinoline-1-carboxylic acid n-propyl ester;
(2R,4S,4aS)-4-[Amino-(3,5-bis(trifluoromethyl-phenyl)-methyl]-2-methyl-6-trifluoromethyl-3,4-dihydro-2H-quinoline-1-carboxylic acid n-propyl ester;
4-[Amino-(3,5-bis(trifluoromethyl-phenyl)-methyl]-2-methyl-6-trifluoromethyl-3,4-dihydro-2H-quinoline-1-carboxylic acid t-butyl ester;
(2R,4R,4aS)-4-[Amino-(3,5-bis(trifluoromethyl-phenyl)-methyl]-2-methyl-6-trifluoromethyl-3,4-dihydro-2H-quinoline-1-carboxylic acid t-butyl ester;
(2R,4S,4aS)-4-[Amino-(3,5-bis(trifluoromethyl-phenyl)-methyl]-2-methyl-6-trifluoromethyl-3,4-dihydro-2H-quinoline-1-carboxylic acid t-butyl ester;
4-[Amino-(3,5-bis(chloro-phenyl)-methyl]-2-ethyl-6-trifluoromethyl-3,4-dihydro-2H-quinoline-1-carboxylic acid ethyl ester;
(2R,4R,4aS)-4-[Amino-(3,5-bis(chloro-phenyl)-methyl]-2-ethyl-6-trifluoromethyl-3,4-dihydro-2H-quinoline-1-carboxylic acid ethyl ester;
(2R,4S,4aS)-4-[Amino-(3,5-bis(chloro-phenyl)-methyl]-2-ethyl-6-trifluoromethyl-3,4-dihydro-2H-quinoline-1-carboxylic acid ethyl ester;
4-[Amino-(3-chloro-5-trifluoromethyl-phenyl)-methyl]-2-ethyl-6-trifluoromethyl-3,4-dihydro-2H-quinoline-1-carboxylic acid ethyl ester;
(2R,4R,4aS)-4-[Amino-(3-chloro-5-trifluoromethyl-phenyl)-methyl]-2-ethyl-6-trifluoromethyl-3,4-dihydro-2H-quinoline-1-carboxylic acid ethyl ester;
(2R,4S,4aS)-4-[Amino-(3-chloro-5-trifluoromethyl-phenyl)-methyl]-2-ethyl-6-trifluoromethyl-3,4-dihydro-2H-quinoline-1-carboxylic acid ethyl ester; and
or a pharmaceutically acceptable salt of said compound.

2. A compound according to claim 1 selected from the group consisting of:
4-[Amino-(3,5-bis-trifluoromethyl-phenyl)-methyl]-2-ethyl-6-trifluoromethyl-3,4-dihydro-2H-quinoline-1-carboxylic acid isopropyl ester;
4-[Amino-(3,5-bis(trifluoromethyl-phenyl)-methyl]-2-ethyl-6-trifluoromethyl-3,4-dihydro-2H-quinoline-1-carboxylic acid n-propyl ester;
4-[Amino-(3,5-bis(trifluoromethyl-phenyl)-methyl]-2-ethyl-6-trifluoromethyl-3,4-dihydro-2H-quinoline-1-carboxylic acid isobutyl ester;
4-[Amino-(3,5-bis(trifluoromethyl-phenyl)-methyl]-2-cyclopropyl-6-trifluoromethyl-3,4-dihydro-2H-quinoline-1-carboxylic acid isopropyl ester;
4-[Amino-(3,5-bis(trifluoromethyl-phenyl)-methyl]-2-methyl-6-trifluoromethyl-3,4-dihydro-2H-quinoline-1-carboxylic acid ethyl ester;
4-[Amino-(3,5-bis(trifluoromethyl-phenyl)-methyl]-2-methyl-6-trifluoromethyl-3,4-dihydro-2H-quinoline-1-carboxylic acid isopropyl ester;
4-[Amino-(3,5-bis(trifluoromethyl-phenyl)-methyl]-2-methyl-6-trifluoromethyl-3,4-dihydro-2H-quinoline-1-carboxylic acid n-propyl ester;
4-[Amino-(3,5-bis(trifluoromethyl-phenyl)-methyl]-2-methyl-6-trifluoromethyl-3,4-dihydro-2H-quinoline-1-carboxylic acid t-butyl ester;
4-[Amino-(3,5-bis(chloro-phenyl)-methyl]-2-ethyl-6-trifluoromethyl-3,4-dihydro-2H-quinoline-1-carboxylic acid ethyl ester;
4-[Amino-(3-chloro-5-trifluoromethyl-phenyl)-methyl]-2-ethyl-6-trifluoromethyl-3,4-dihydro-2H-quinoline-1-carboxylic acid ethyl ester;
or a pharmaceutically acceptable salt of said compound.

3. A compound according to claim 1 selected from the group consisting of:
(2R,4R,4aS)-4-[Amino-(3,5-bis-trifluoromethyl-phenyl)-methyl]-2-ethyl-6-trifluoromethyl-3,4-dihydro-2H-quinoline-1-carboxylic acid isopropyl ester;
(2R,4R,4aS)-4-[Amino-(3,5-bis(trifluoromethyl-phenyl)-methyl]-2-ethyl-6-trifluoromethyl-3,4-dihydro-2H-quinoline-1-carboxylic acid n-propyl ester;
(2R,4R,4aS)-4-[Amino-(3,5-bis(trifluoromethyl-phenyl)-methyl]-2-ethyl-6-trifluoromethyl-3,4-dihydro-2H-quinoline-1-carboxylic acid isobutyl ester;
(2S,4R,4aS)-4-[Amino-(3,5-bis(trifluoromethyl-phenyl)-methyl]-2-cyclopropyl-6-trifluoromethyl-3,4-dihydro-2H-quinoline-1-carboxylic acid isopropyl ester;
(2R,4R,4aS)-4-[Amino-(3,5-bis(trifluoromethyl-phenyl)-methyl]-2-methyl-6-trifluoromethyl-3,4-dihydro-2H-quinoline-1-carboxylic acid ethyl ester;
(2R,4R,4aS)-4-[Amino-(3,5-bis(trifluoromethyl-phenyl)-methyl]-2-methyl-6-trifluoromethyl-3,4-dihydro-2H-quinoline-1-carboxylic acid isopropyl ester;
(2R,4R,4aS)-4-[Amino-(3,5-bis(trifluoromethyl-phenyl)-methyl]-2-methyl-6-trifluoromethyl-3,4-dihydro-2H-quinoline-1-carboxylic acid n-propyl ester;
(2R,4R,4aS)-4-[Amino-(3,5-bis(trifluoromethyl-phenyl)-methyl]-2-methyl-6-trifluoromethyl-3,4-dihydro-2H-quinoline-1-carboxylic acid t-butyl ester;
(2R,4R,4aS)-4-[Amino-(3,5-bis(chloro-phenyl)-methyl]-2-ethyl-6-trifluoromethyl-3,4-dihydro-2H-quinoline-1-carboxylic acid ethyl ester;
(2R,4R,4aS)-4-[Amino-(3-chloro-5-trifluoromethyl-phenyl)-methyl]-2-ethyl-6-trifluoromethyl-3,4-dihydro-2H-quinoline-1-carboxylic acid ethyl ester;
or a pharmaceutically acceptable salt of said compound.

4. A method for treating atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal by administering to a mammal in need of such treatment an atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction treating amount of a compound of claim 1 or a pharmaceutically acceptable salt of said compound.

5. A pharmaceutical composition which comprises a therapeutically effective amount of a compound of claim 1 or a pharmaceutically acceptable salt of said compound and a pharmaceutically acceptable vehicle, diluent or carrier.

6. A pharmaceutical composition for the treatment of atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal which comprises a therapeutically effective amount of a compound of claim 1 or a pharmaceutically acceptable salt of said compound and a pharmaceutically acceptable vehicle, diluent or carrier.

7. A pharmaceutical combination composition comprising: a therapeutically effective amount of a composition comprising
a first compound, said first compound being a compound of claim 1 or a pharmaceutically acceptable salt of said compound;
a second compound, said second compound being an HMG CoA reductase inhibitor, an MTP/Apo B secretion inhibitor, a PPAR modulator, a bile acid reuptake inhibitor, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant; and
a pharmaceutical vehicle, diluent or carrier.

8. A pharmaceutical combination composition as recited in claim 7 wherein the second compound is an HMG-CoA reductase inhibitor or a PPAR modulator.

9. A pharmaceutical combination composition as recited in claim 8 wherein the second compound is lovastatin, simvastatin, pravastatin, fluvastatin, atorvastatin, rivastatin, rosuvastatin or pitavastatin.

10. A method for treating atherosclerosis in a mammal comprising administering to a mammal in need of treatment thereof;
a first compound, said first compound being a compound of claim 1 or a pharmaceutically acceptable salt of said compound; and
a second compound, said second compound being an HMG CoA reductase inhibitor, a PPAR modulator, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant
wherein the amounts of first and second compounds result in a therapeutic effect.

11. A method for treating atherosclerosis as recited in claim 10 wherein the second compound is an HMG-CoA reductase inhibitor or a PPAR modulator.

12. A method for treating atherosclerosis as recited in claim 11 wherein the second compound is lovastatin, simvastatin, pravastatin, fluvastatin, atorvastatin, rivastatin, rosuvastatin or pitavastatin.

13. A kit for achieving a therapeutic effect in a mammal comprising packaged in association a first therapeutic agent comprising a therapeutically effective amount of a compound of claim 1 or a pharmaceutically acceptable salt of said compound and a pharmaceutically acceptable carrier, a second therapeutic agent comprising a therapeutically effective amount of an HMG CoA reductase inhibitor, a PPAR modulator, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant and a pharmaceutically acceptable carrier and directions for administration of said first and second agents to achieve the therapeutic effect.

14. A kit as recited in claim 13 wherein said second therapeutic agent comprises an HMG-CoA reductase inhibitor or a PPAR modulator.

15. A kit as recited in claim 14 wherein said second therapeutic agent comprises lovastatin, simvastatin, pravastatin, fluvastatin, atorvastatin, rivastatin, rosuvastatin or pitavastatin.

16. A compound (2R,4R,4aS)-4-[Amino-(3,5-bis-trifluoromethyl-phenyl)-methyl]-2-ethyl-6-trifluoromethyl-3,4-dihydro-2H-quinoline-1-carboxylic acid isopropyl ester or a pharmaceutically acceptable salt of said compound.

17. A method for treating atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal by administering to a mammal in need of such treatment an atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction treating amount of a compound of claim 16 or a pharmaceutically acceptable salt of said compound.

18. A pharmaceutical combination composition comprising: a therapeutically effective amount of a composition comprising
a first compound, said first compound being a compound of claim 16 or a pharmaceutically acceptable salt of said compound;
a second compound, said second compound being an HMG CoA reductase inhibitor, an MTP/Apo B secretion inhibitor, a PPAR modulator, a bile acid reuptake inhibitor, a cholesterol absorption inhibitor, a cholesterol synthesis inhibitor, a fibrate, niacin, slow-release niacin, a combination of niacin and lovastatin, an ion-exchange resin, an antioxidant, an ACAT inhibitor or a bile acid sequestrant; and
a pharmaceutical vehicle, diluent or carrier.

19. A pharmaceutical combination composition as recited in claim 18 wherein the second compound is lovastatin, simvastatin, pravastatin, fluvastatin, atorvastatin, rivastatin, rosuvastatin or pitavastatin or a pharmaceutically acceptable salt thereof.

20. A pharmaceutical combination composition comprising: a therapeutically effective amount of a composition comprising
(2R,4R,4aS)-4-[Amino-(3,5-bis-trifluoromethyl-phenyl)-methyl]-2-ethyl-6-trifluoromethyl-3,4-dihydro-2H-quinoline-1-carboxylic acid isopropyl ester or a pharmaceutically acceptable salt of said compound;
atorvastatin, or a pharmaceutically acceptable salt of said compound; and
a pharmaceutical vehicle, diluent or carrier.
